(19) United States
(12) Patent Application Publication
(10) Pub. No.: US 2009/0270382 A1
(43) Pub. Date: Oct. 29, 2009

(54) Soluble epoxide hydrolase inhibitors

(75) Inventors: Bhasker R. Aavula, Pleasanton, CA (US); Sampath-Kumar Anandan, Fremont, CA (US); Richard D. Gless, JR., Oakland, CA (US)

Correspondence Address: FOLEY & LARDNER LLP, 975 PAGE MILL ROAD, PALO ALTO, CA, 94304, US

(73) Assignee: Arete Therapeutics, Inc.

(21) Appl. No.: 12/426,136

(22) Filed: Apr. 17, 2009

Related U.S. Application Data
(60) Provisional application No. 61/046,316, filed on Apr. 18, 2008.

(51) Int. Cl. A61K 31/5365; C07D 213/02; A61K 31/4545; C12N 9/99; C07D 413/14; A61K 31/5377; C07D 498/02; A61P 9/12; A61P 29/00; A61P 11/00; A61P 3/10

(52) U.S. Cl. 514/230.5; 546/194; 514/318; 435/184; 544/129; 514/235.5; 544/105

(57) ABSTRACT

Disclosed are amide, thioamide, urea and thiourea compounds and compositions that inhibit soluble epoxide hydrolase (sEH), methods for preparing the compounds and compositions, and methods for treating patients with such compounds and compositions. The compounds, compositions, and methods are useful for treating a variety of sEH mediated diseases, including hypertensive, cardiovascular, inflammatory, and diabetic-related diseases.

### CROSS-REFERENCE TO RELATED APPLICATION

This application claims the benefit under 35 U.S.C. § 119(e) of U.S. Provisional Patent Application Ser. No. 61/046,316, filed on Apr. 18, 2008, which is incorporated herein by reference in its entirety.

### BACKGROUND OF THE INVENTION

1. Field of the Invention

This invention relates to the field of pharmaceutical chemistry. Provided herein are amide, thioamide, urea, and thiourea compounds that inhibit soluble epoxide hydrolase (sEH), pharmaceutical compositions containing such compounds, methods for preparing the compounds and formulations, and methods for treating patients with such compounds and compositions. The compounds, compositions, and methods are useful for treating a variety of diseases, including hypertensive, cardiovascular, inflammatory diseases, metabolic syndrome, smooth muscle disorders, and diabetic-related diseases.

2. State of the Art

The arachidonate cascade is a ubiquitous lipid signaling cascade in which arachidonic acid is liberated from the plasma membrane lipid reserves in response to a variety of extra-cellular and/or intra-cellular signals. The released arachidonic acid is then available to act as a substrate for a variety of oxidative enzymes that convert arachidonic acid to signaling lipids that play critical roles in, for example, inflammation and other disease conditions. Disruption of the pathways leading to the lipids remains an important strategy for many commercial drugs used to treat a multitude of inflammatory disorders. For example, non-steroidal anti-inflammatory drugs (NSAIDs) disrupt the conversion of arachidonic acid to prostaglandins by inhibiting cyclooxygenases (COX1 and COX2). New asthma drugs, such as SINGULAIR™ disrupt the conversion of arachidonic acid to leukotrienes by inhibiting lipoxygenase (LOX). Certain cytochrome P450-dependent enzymes convert arachidonic acid into a series of epoxide derivatives known as epoxyeicosatrienoic acids (EETs). These EETs are particularly prevalent in the vascular endothelium (cells that make up arteries and vascular beds), kidney, and lung. In contrast to many of the end products of the prostaglandin and leukotriene pathways, the EETs have a variety of anti-inflammatory and anti-hypertensive properties and are known to be potent vasodilators and mediators of vascular permeability.

While EETs have potent effects in vivo, the epoxide moiety of the EETs is rapidly hydrolyzed into the less active dihydroxyeicosatrienoic acid (DHET) form by an enzyme called soluble epoxide hydrolase (sEH). Inhibition of sEH has been found to significantly reduce blood pressure in hypertensive animals (see, e.g., Yu et al. Circ. Res. 87:992-8 (2000) and Sinal et al. J. Biol. Chem. 275:40504-10 (2000)), to reduce the production of proinflammatory nitric oxide (NO), cytokines, and lipid mediators, and to contribute to inflammatory resolution by enhancing lipoxin A₄ production in vivo (see. Schmelzer et al. Proc. Nat'l Acad. Sci. USA 102(28):9772-7 (2005)).

Various small molecule compounds have been found to inhibit sEH and elevate EET levels (Morisseau et al. Annu. Rev. Pharmacol. Toxicol. 45:311-33 (2005)). The availability of more potent compounds capable of inhibiting sEH and its inactivation of EETs would be highly desirable for treating a wide range of disorders that are mediated by conversion of sEH to EET's including inflammation and hypertension.

### SUMMARY OF THE INVENTION

This invention relates to compounds and their pharmaceutical compositions, to their preparation, and to their uses for treating diseases mediated by soluble epoxide hydrolase (sEH). In accordance with one aspect of the invention, provided are compounds having Formula (I) or a pharmaceutically acceptable salt thereof:

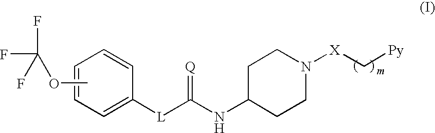

wherein

Q is O or S;

L is a covalent bond, —NH— or —CR¹R²—; where R¹ and R² are independently hydrogen or alkyl or R¹ and R² together with the carbon atom bound thereto form a C₃-C₆ cycloalkyl ring;

Py is pyridyl or substituted pyridyl;

X is —C(O)—, or —SO₂—; and

m is 0, 1, or 2; and

wherein when m is 0 and Q is O, then X is on the 3- or 4-position of the pyridyl ring.

In another embodiment, provided are compounds of Table 1 or a pharmaceutically acceptable salt thereof.

In accordance with another aspect of the invention, provided is a pharmaceutical composition comprising a pharmaceutically acceptable carrier and a therapeutically effective amount of a compound of the invention or a pharmaceutically acceptable salt thereof.

In accordance with another aspect of the invention, provided is a method for treating a soluble expoxide hydrolase mediated disease, said method comprising administering to a patient a pharmaceutical composition comprising a pharmaceutically acceptable carrier and a therapeutically effective amount of a compound of the invention or a pharmaceutically acceptable salt thereof.

In accordance with yet another aspect of the invention, provided is a method for inhibiting a soluble expoxide hydrolase, said method comprising contacting the soluble epoxide hydrolase with an effective amount of a compound of the invention or a pharmaceutically acceptable salt thereof.

These and other embodiments of the invention are further described in the text that follows.

### DETAILED DESCRIPTION OF THE INVENTION

### Definitions

As used herein, the following definitions shall apply unless otherwise indicated.

“cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The term “soluble epoxide hydrolase” (“sEH”) as used herein includes all functional genetic variants. The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH and the nucleic acid sequence encoding the human sEH are set forth in U.S. Pat. No. 5,445,956. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

“Chronic Obstructive Pulmonary Disease” or “COPD” is also sometimes known as “chronic obstructive airway disease”, “chronic obstructive lung disease”, and “chronic airways disease.” COPD is generally defined as a disorder characterized by reduced maximal expiratory flow and slow forced emptying of the lungs. COPD is considered to encompass two related conditions, emphysema and chronic bronchitis. COPD can be diagnosed by the general practitioner using art recognized techniques, such as the patient's forced vital capacity (“FVC”), the maximum volume of air that can be forcibly expelled after a maximal inhalation. In the offices of general practitioners, the FVC is typically approximated by a 6 second maximal exhalation through a spirometer. The definition, diagnosis and treatment of COPD, emphysema, and chronic bronchitis are well known in the art and discussed in detail by, for example, Honig and Ingram, in Harrison's Principles of Internal Medicine, (Fauci et al., Eds), 14th Ed., 1998, McGraw-Hill, New York, pp. 1451-1460 (hereafter, “Harrison's Principles of Internal Medicine”). As the names imply, “obstructive pulmonary disease” and “obstructive lung disease” refer to obstructive diseases, as opposed to restrictive diseases. These diseases particularly include COPD, bronchial asthma, and small airway disease.

“Emphysema” is a disease of the lungs characterized by permanent destructive enlargement of the airspaces distal to the terminal bronchioles without obvious fibrosis.

“Chronic bronchitis” is a disease of the lungs characterized by chronic bronchial secretions which last for most days of a month, for three months, a year, for two years, etc.

“Small airway disease” refers to diseases where airflow obstruction is due, solely or predominantly to involvement of the small airways. These are defined as airways less than 2 mm in diameter and correspond to small cartilaginous bronchi, terminal bronchioles, and respiratory bronchioles. Small airway disease (SAD) represents luminal obstruction by inflammatory and fibrotic changes that increase airway resistance. The obstruction may be transient or permanent.

“Interstitial lung diseases (ILDs)” are restrictive lung diseases involving the alveolar walls, perialveolar tissues, and contiguous supporting structures. As discussed on the website of the American Lung Association, the tissue between the air sacs of the lung is the interstitium, and this is the tissue affected by fibrosis in the disease. Persons with such restrictive lung disease have difficulty breathing in because of the stiffness of the lung tissue but, in contrast to persons with obstructive lung disease, have no difficulty breathing out. The definition, diagnosis and treatment of interstitial lung diseases are well known in the art and discussed in detail by, for example, Reynolds, H. Y., in Harrison's Principles of Internal Medicine, supra, at pp. 1460-1466. Reynolds notes that, while ILDs have various initiating events, the immunopathological responses of lung tissue are limited and the ILDs therefore have common features.

“Idiopathic pulmonary fibrosis,” or “IPF,” is considered the prototype ILD. Although it is idiopathic in that the cause is not known, Reynolds, supra, notes that the term refers to a well defined clinical entity.

“Bronchoalveolar lavage,” or “BAL,” is a test which permits removal and examination of cells from the lower respiratory tract and is used in humans as a diagnostic procedure for pulmonary disorders such as IPF. In human patients, it is usually performed during bronchoscopy.

“Diabetic neuropathy” refers to acute and chronic peripheral nerve dysfunction resulting from diabetes.

“Diabetic nephropathy” refers to renal diseases resulting from diabetes.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5, preferably 1 to 3, or more preferably 1 to 2 substituents selected from the group consisting of alkoxy, substituted alkoxy, amino, substituted amino, aryl, substituted aryl, carboxyl, carboxyl ester, cyano, cycloalkyl, substituted cycloalkyl, halo, hydroxy, heteroaryl, substituted heteroaryl, heterocyclic, substituted heterocyclic, and nitro, wherein said substituents are defined herein.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, cycloalkyl, substituted cycloalkyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR³⁴C(O)alkyl, —NR³⁴C(O) substituted alkyl, —NR³⁴C(O)cycloalkyl, —NR³⁴C(O) substituted cycloalkyl, —NR³⁴C(O)aryl, —NR³⁴C(O) substituted aryl, —NR³⁴C(O)heteroaryl, —NR³⁴C(O) substituted heteroaryl, —NR³⁴C(O)heterocyclic, and —NR³⁴C(O) substituted heterocyclic wherein R³⁴ is hydrogen or alkyl and wherein alkyl, substituted alkylcycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR¹⁸R¹⁹ where R¹⁸ and R¹⁹ are independently selected from the group consisting of hydrogen, alkyl, substituted alkyl, aryl, substituted aryl, cycloalkyl, substituted cycloalkyl, heteroaryl, substituted heteroaryl, heterocyclic, substituted heterocyclic, and wherein R¹⁸ and R¹⁹ are optionally joined, together with the nitrogen bound thereto to form a heterocyclic or substituted heterocyclic group, provided that R¹⁸ and R¹⁹ are both not hydrogen. When R¹⁸ is hydrogen and R¹⁹ is alkyl, the substituted amino group is sometimes referred to herein as alkylamino. When R¹⁸ and R¹⁹ are alkyl, the substituted amino group is sometimes referred to herein as dialkylamino. When referring to a monosubstituted amino, it is meant that either R¹⁸ or R¹⁹ is hydrogen but not both. When referring to a disubstituted amino, it is meant that neither R¹⁸ nor R¹⁹ are hydrogen.

“Aminocarbonyl” refers to the group —C(O)NR¹⁰R¹¹ where R¹⁰ and R¹¹ are independently selected from the group consisting of hydrogen, alkyl, substituted alkyl, aryl, substituted aryl, cycloalkyl, substituted cycloalkyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of acyl, acylamino, alkyl, substituted alkyl, alkoxy, substituted alkoxy, aryloxy, substituted sulfonyl, amino, substituted amino, aminocarbonyl, aryl, substituted aryl, carboxyl, carboxyl ester, (carboxyl ester)amino, cyano, cycloalkyl, substituted cycloalkyl, halo, hydroxyl, heteroaryl, substituted heteroaryl, heterocyclic, substituted heterocyclic, and nitro wherein said substituents are defined herein.

“Aryloxy” refers to the group —O-aryl, where aryl is as defined herein, that includes, by way of example, phenoxy and naphthoxy.

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic.

“(Carboxyl ester)amino” refers to the group —NR¹⁴—C(O)O-alkyl, —NR¹⁴—C(O)O-substituted alkyl, —NR¹⁴—C(O)O-aryl, —NR¹⁴—C(O)O-substituted aryl, —NR¹⁴—C(O)O-cycloalkyl, —NR¹⁴—C(O)O-substituted cycloalkyl, —NR¹⁴—C(O)O-heteroaryl, —NR¹⁴—C(O)O-substituted heteroaryl, —NR¹⁴—C(O)O-heterocyclic, and —NR¹⁴—C(O)O-substituted heterocyclic wherein R¹⁴ is alkyl or hydrogen, and wherein alkyl, substituted alkyl, cycloalkyl, substituted cycloalkyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spirobicyclo groups such as spiro[4.5]dec-8-yl:

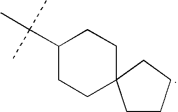

“Substituted cycloalkyl” refers to a cycloalkyl group having from 1 to 5 or preferably 1 to 3 substituents selected from the group consisting of oxo, thione, alkyl, substituted alkyl, alkoxy, substituted alkoxy, amino, substituted amino, aryl, substituted aryl, carboxyl, carboxyl ester, cyano, cycloalkyl, substituted cycloalkyl, halo, hydroxyl, heteroaryl, substituted heteroaryl, heterocyclic, substituted heterocyclic, and nitro, wherein said substituents are defined herein.

“Substituted sulfonyl” refers to the group —SO₂-alkyl, —SO₂-substituted alkyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, cycloalkyl, substituted cycloalkyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl.

“Halo” or “halogen” refers to fluoro, chloro, bromo and iodo and preferably is fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, and/or sulfonyl moieties. Preferred heteroaryls include pyridinyl (also referred to as pyridyl), pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Substituted pyridyl” refers to pyridyl substituted with from 1 to 4, or preferably 1 to 2 substituents independently selected from the group consisting of the same group of substituents defined for substituted aryl. As used herein, substituted pyridyl also includes pyridyl with two substituents on two adjacent carbon atoms joined together to form an optionally substituted heterocyclic group fused with the pyridyl ring. An example is shown below where two substituents on two adjacent carbon atoms join together to form a methyl substituted heterocyclic group fused with the pyridyl ring:

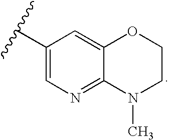

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic heterocyclic ring. In one embodiment, the nitrogen and/or sulfur atom(s) of the heterocyclic group are optionally oxidized to provide for the N-oxide, sulfinyl, and/or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl. Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Thione” refers to the atom (═S).

“Compound” or “compounds” as used herein is meant to include the stereoisomers and tautomers of the indicated formulas.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, aluminum, lithium and ammonium, for example tetraalkylammonium, and the like when the molecule contains an acidic functionality; and when the molecule contains a basic functionality, salts of organic or inorganic acids, such as hydrochloride, sulfate, phosphate, diphosphate, nitrate hydrobromide, tartrate, mesylate, acetate, malate, maleate, fumarate, tartrate, succinate, citrate, lactate, pamoate, salicylate, stearat, methanesulfonate, p-toluenesulfonate, and oxalate, and the like. Suitable pharmaceutically acceptable salts include those listed in Remington's Pharmaceutical Sciences, 17th Edition, pg. 1418 (1985) and P. Heinrich Stahl, Camille G. Wermuth (Eds.), Handbook of Pharmaceutical Salts Properties, Selection, and Use; 2002. Examples of acid addition salts include those formed from acids such as hydroiodic, phosphoric, metaphosphoric, nitric and sulfuric acids, and with organic acids, such as alginic, ascorbic, anthranilic, benzoic, camphorsulfuric, citric, embonic (pamoic), ethanesulfonic, formic, fumaric, furoic, galacturonic, gentisic, gluconic, glucuronic, glutamic, glycolic, isonicotinic, isothionic, lactic, malic, mandelic, methanesulfonic, mucic, pantothenic, phenylacetic, propionic, saccharic, salicylic, stearic, succinic, sulfinilic, trifluoroacetic and arylsulfonic for example benzenesulfonic and p-toluenesulfonic acids. Examples of base addition salts formed with alkali metals and alkaline earth metals and organic bases include chloroprocaine, choline, N,N-dibenzylethylenediamine, diethanolamine, tromethamine, ethylenediamine, lysine, meglumaine (N-methylglucamine), and procaine, as well as internally formed salts. Salts having a non-physiologically acceptable anion or cation are within the scope of the invention as useful intermediates for the preparation of physiologically acceptable salts and/or for use in non-therapeutic, for example, in vitro, situations.

“Treating” or “treatment” of a disease in a patient refers to (1) preventing the disease from occurring in a patient that is predisposed or does not yet display symptoms of the disease; (2) inhibiting the disease or arresting its development; or (3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc) are not intended for inclusion herein. In such cases, the maximum number of such substitutions is three. For example, serial substitutions of substituted aryl groups with two other substituted aryl groups are limited to -substituted aryl-(substituted aryl)-substituted aryl. It is also understood that in all substituted groups defined above, polymers arrived at by defining substituents with other substituents (e.g., substituted aryl having a substituted alkyl group as a substituent which is itself substituted with a substituted aryl group, etc.) are not intended to include cases where the maximum number of such substituents exceeds five. That is to say that each of the above definitions is constrained by a limitation that substitutions do not exceed five, for example, substituted aryl groups are limited to -substituted aryl-(substituted alkyl)-(substituted cycloalkyl)-(substituted alkyl)-(substituted alkyl).

Similarly, it is understood that the above definitions are not intended to include impermissible substitution patterns (e.g., methyl substituted with 5 fluoro groups). Such impermissible substitution patterns are well known to the skilled artisan.

Accordingly, the present invention provides a compound of Formula (I) or a pharmaceutically acceptable salt thereof:

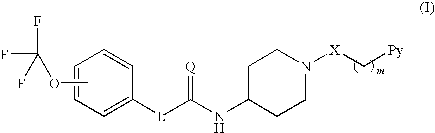

wherein

Q is O or S;

L is a covalent bond, —NH— or —CR¹R²—; where R¹ and R² are independently hydrogen or alkyl or R¹ and R² together with the carbon atom bound thereto form a C₃-C₆ cycloalkyl ring;

Py is pyridyl or substituted pyridyl;

X is —C(O)—, or —SO₂—; and

m is 0, 1, or 2; and

wherein when m is 0 and Q is O, then X is on the 3- or 4-position of the pyridyl ring.

Various embodiments relating to the compounds or pharmaceutically acceptable salts of Formula (I) are listed below. These embodiments can be combined with each other or with any other embodiments described in this application. In some aspects, provided are compounds of Formula (I) having one or more of the following features.

In some embodiments, the group —OCF₃ is para to L. In some embodiments, the group —OCF₃ is meta to L. In some embodiments, the group —OCF₃ is ortho to L.

In some embodiments, L is —NH—.

In some embodiments, L is —CR¹R²— where R¹ and R² are independently H or alkyl or R¹ and R² together with the carbon atom bound thereto form a C₃-C₆ cycloalkyl ring. In some embodiments, L is —CH₂—.

In some embodiments, L is a covalent bond.

In some embodiments, X is —C(O)—. In some embodiments, X is —SO₂—.

In some embodiments, Q is O. In some embodiments, Q is S.

In some embodiments, m is 0. In some embodiments, m is 1.

In some embodiments, Py is pyridyl.

In some embodiments, Py is substituted pyridyl. In some embodiment, Py is pyridyl substituted with from 1 to 4 substituents independently selected from the group consisting of halo, alkyl, substituted alkyl, aryloxy, substituted sulfonyl, acyl, acylamino, aminocarbonyl, (carboxyl ester)amino, cycloalkyl, substituted cycloalkyl, aryl, substituted aryl, heterocyclic, substituted heterocyclic, alkoxy, substituted alkoxy, carboxy, carboxyl ester, cyano, and nitro. In some embodiments, the pyridyl is substituted with 1 or 2 substituents. In some embodiments, the substituents are independently selected from the group consisting of fluoro, chloro, methyl, trifluoromethyl, methoxy, trifluoromethoxy, and carboxy.

In some embodiments, provided is a compound having Formula (IIa) or (IIb) or a pharmaceutically acceptable salt thereof:

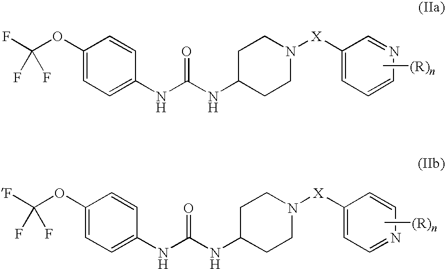

wherein

X is —C(O)—, or —SO₂—;

each R is independently selected from the group consisting of halo, alkyl, substituted alkyl, aryloxy, substituted sulfonyl, acyl, acylamino, aminocarbonyl, (carboxyl ester)amino, carboxyl, carboxyl ester, cycloalkyl, substituted cycloalkyl, aryl, substituted aryl, heterocyclic, substituted heterocyclic, alkoxy, substituted alkoxy, cyano, and nitro; or two R groups on two adjacent pyridyl carbon atoms join together to form an optionally substituted heterocyclic group fused with the pyridyl ring; and

n is 0, 1, 2, 3, or 4.

In some embodiments, provided is a compound having Formula (IIIa) or (IIIb) or a pharmaceutically acceptable salt thereof:

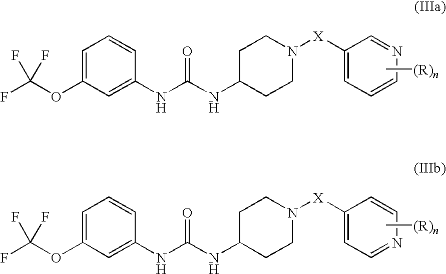

wherein

X is —C(O)—, or —SO₂—;

each R is independently selected from the group consisting of halo, alkyl, substituted alkyl, cycloalkyl, substituted cycloalkyl, aryloxy, substituted sulfonyl, acyl, acylamino, aminocarbonyl, (carboxyl ester)amino, aryl, substituted aryl, heterocyclic, substituted heterocyclic, alkoxy, substituted alkoxy, carboxy, carboxyl ester, cyano, and nitro; or two R groups on two adjacent pyridyl carbon atoms join together to form an optionally substituted heterocyclic group fused with the pyridyl ring; and

n is 0, 1, 2, 3, or 4.

In some embodiments, provided is a compound having Formula (IVa), (IVb) or (IVc) or a pharmaceutically acceptable salt thereof:

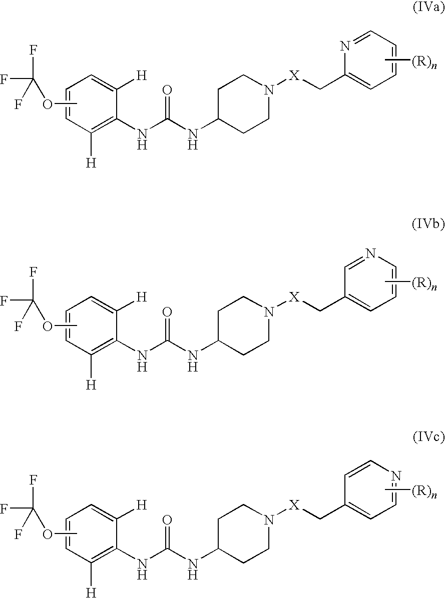

wherein

X is —C(O)—, or —SO₂—;

each R is independently selected from the group consisting of halo, alkyl, substituted alkyl, aryloxy, substituted sulfonyl, acyl, acylamino, aminocarbonyl, (carboxyl ester)amino, carboxy, carboxyl ester, cycloalkyl, substituted cycloalkyl, aryl, substituted aryl, heterocyclic, substituted heterocyclic, alkoxy, substituted alkoxy, cyano, and nitro; or two R groups on two adjacent pyridyl carbon atoms join together to form an optionally substituted heterocyclic group fused with the pyridyl ring; and

n is 0, 1, 2, 3, or 4.

Various embodiments relating to the compounds or pharmaceutically acceptable salts of Formulas (IIa), (IIb), (IIIa), (IIIb), (IVa), (IVb), and (IVc) are listed below. These embodiments can be combined with each other or with any other embodiments described in this application. In some aspects, provided are compounds of Formulas (IIa), (IIb), (IIIa), (IIIb), (IVa), (IVb), and (IVc) having one or more of the following features.

In some embodiments, X is —CO—. In some embodiments X is —SO₂—.

In some embodiments, Q is O. In some embodiments Q is S.

In some embodiments, n is 0. In some embodiments, n is 1. In some embodiments, n is 2.

In some embodiments, R is independently selected from the group consisting of halo, alkyl, substituted alkyl, alkoxy, substituted alkoxy, aryloxy, substituted sulfonyl, acylamino, aminocarbonyl, (carboxyl ester)amino, acyl, carboxyl, carboxyl ester, cyano, and nitro. In some aspects, R is selected from the group consisting of fluoro, chloro, methyl, trifluoromethyl, methoxy, trifluoromethoxy, phenyl, phenoxy, and carboxy. In some embodiments, R is aryl or heterocyclic. In some embodiments, R is phenyl or morpholino. In some embodiments, two R groups on two adjacent carbon atoms join together to form an optionally substituted heterocyclic ring fused with the pyridyl ring. In some embodiments, the heterocyclic ring is substituted with alkyl. In some embodiments, the heterocyclic ring is a morpholino optionally substituted with alkyl.

In some embodiments, provided is a compound selected from Table 1 or a pharmaceutically acceptable salt thereof.

TABLE 1

| Compound # | structure | name |
|---|---|---|
| 1 | 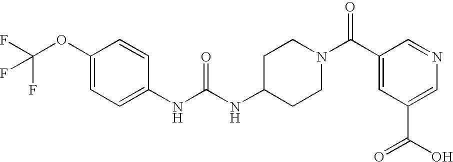 | 5-(4-(3 -(4- (trifluoromethoxy)phenyl) ureido)piperidine-1- carbonyl)nicotinic acid |
| 2 | 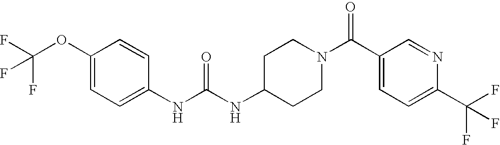 | 1-(4-(trifluoromethoxy) phenyl)-3-(1-(6- (trifluoromethyl)nicotinoyl) piperidin-4-yl)urea |
| 3 | 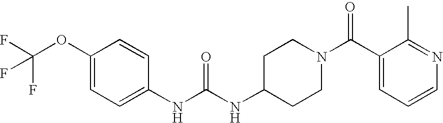 | 1-(1-(2- methylnicotinoyl) piperidin-4-yl)-3-(4- (trifluoromethoxy)phenyl)urea |
| 4 | 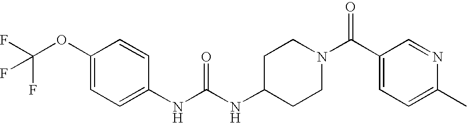 | 1-(1-(6- methylnicotinoyl) piperidin-4-yl)-3-(4- (trifluoromethoxy)phenyl)urea |
| 5 | 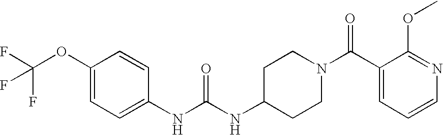 | 1-(1-(2- methoxynicotinoyl) piperidin-4-yl)-3-(4- (trifluoromethoxy)phenyl)urea |
| 6 | 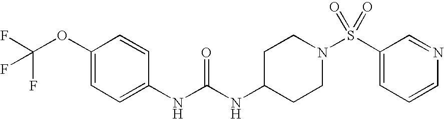 | 1-(1-(pyridin-3- ylsulfonyl)piperidin-4-yl)-3-(4- (trifluoromethoxy)phenyl)urea |
| 7 | 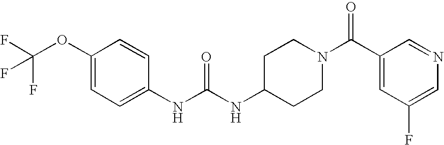 | 1-(1-(5- fluoronicotinoyl)piperidin-4- yl)-3-(4- (trifluoromethoxy)phenyl)urea |
| 8 | 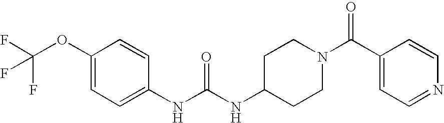 | 1-(1-isonicotinoylpiperidin-4- yl)-3-(4- (trifluoromethoxy)phenyl)urea |
| 9 | 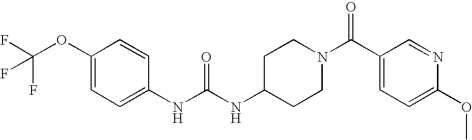 | 1-(1-(6- methoxynicotinoyl) piperidin-4-yl)-3-(4- (trifluoromethoxy)phenyl)urea |
| 10 | 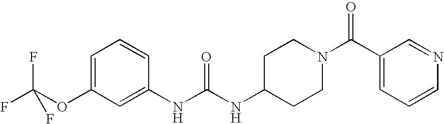 | 1-(1-nicotinoylpiperidin-4-yl)- 3-(3- (trifluoromethoxy)phenyl)urea |
| 11 | 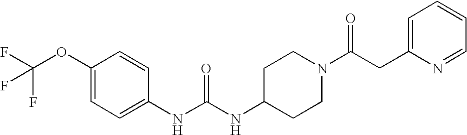 | 1-(1-(2-(pyridin-2- yl)acetyl)piperidin-4-yl)-3-(4- (trifluoromethoxy)phenyl)urea |
| 12 | 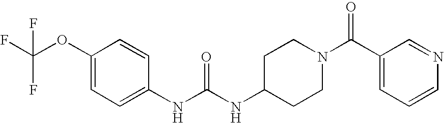 | 1-(1-nicotinoylpiperidin-4-yl)- 3-(4- (trifluoromethoxy)phenyl)urea |
| 13 | 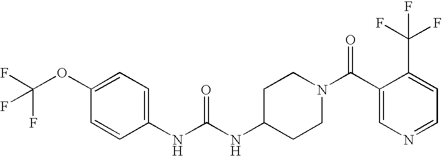 | 1-(4-(trifluoromethoxy)phenyl)- 3-(1-(4- (trifluoromethyl)nicotinoyl) piperidin-4-yl)urea |
| 14 | 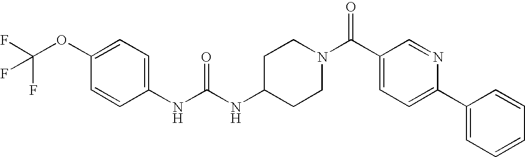 | 1-(1-(6- phenylnicotinoyl)piperidin-4- yl)-3-(4- (trifluoromethoxy)phenyl)urea |
| 15 | 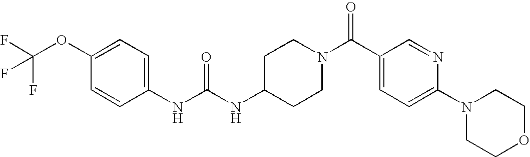 | 1-(1-(6- morpholinonicotinoyl)pipendin- 4-yl)-3-(4- (trifluoromethoxy)phenyl)urea |
| 16 | 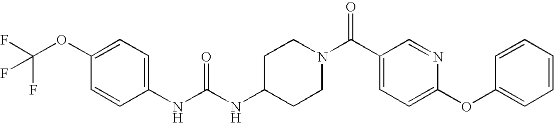 | 1-(1-(6- phenoxynicotinoyl)piperidin-4- yl)-3-(4- (trifluoromethoxy)phenyl)urea |
| 17 | 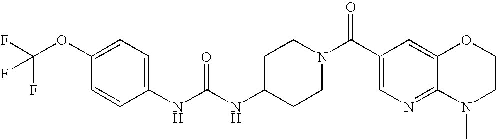 | 1-(1-(4-methyl-3,4-dihydro-2H- pyrido[3,2-b][1,4]oxazine-7- carbonyl)piperidin-4-yl)-3-(4- (trifluoromethoxy)phenyl)urea |
| 18 | 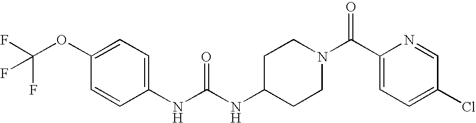 | 1-(1-(5- chloropicolinoyl)piperidin-4- yl)-3-(4- (trifluoromethoxy)phenyl)urea |

It is contemplated that compounds of this invention with a trifluoromethoxyphenyl moiety in conjunction with a pyridyl or substituted pyridyl moiety have improved pharmacokinetic profile as compared to compounds having a cycloalkyl group in place of the trifluoromethoxyphenyl moiety or as compared to compounds having an alkyl, a phenyl or substituted phenyl moiety in place of the pyridyl or substituted pyridyl moiety.

In one embodiment, provided is a pharmaceutical composition comprising a pharmaceutically acceptable carrier and a therapeutically effective amount of a compound or pharmaceutically acceptable salt of any one of Formulas (I), (IIa), (IIb), (IIIa), (IIIb), (IVa), (IVb), and (IVc) or of Table 1 for treating a soluble expoxide hydrolase mediated disease.

In another embodiment, provided is a method for treating a soluble expoxide hydrolase mediated disease, said method comprising administering to a patient a pharmaceutical composition comprising a pharmaceutically acceptable carrier and a therapeutically effective amount of a compound or pharmaceutically acceptable salt of any one of formulas (I), (IIa), (IIb), (IIIa), (IIIb), (IVa), (IVb), and (IVc) or of Table 1. Soluble expoxide hydrolase mediated diseases or conditions include but are not limited to hypertension; inflammation, such as renal inflammation, hepatic inflammation, vascular inflammation, and lung inflammation; adult respiratory distress syndrome; diabetic complications; endothelial disfunction; metabolic syndrome; diabetes; diabetic complications; arthritis; end stage renal disease; nephropathy; kidney malfunction, such as proteinuria, and in particular, albuminuria, and subsequent edema resulting therefrom, macrophage infiltration, and the like; proliferation of vascular smooth muscle cells, such as (atherosclerosis, stenosis, restenosis); damages from stroke; atherosclerosis; disorders; such as high intraocular pressure, dry eye syndrome, age-related macular degeneration, diabetic retinopathy, glaucoma, and rejection of a corneal graft; cadiomyopathy; migraine; and other diseases or disorders described herein.

It has previously been shown that inhibitors of soluble epoxide hydrolase (“sEH”) can reduce hypertension (see, e.g., U.S. Pat. No. 6,531,506). Such inhibitors can be useful in controlling the blood pressure of persons with undesirably high blood pressure, including those who suffer from diabetes.

In some embodiments, compounds of the invention are administered to a subject in need of treatment for hypertension, specifically renal, hepatic, or pulmonary hypertension; inflammation, specifically renal inflammation, hepatic inflammation, vascular inflammation, and lung inflammation; adult respiratory distress syndrome; diabetic complications; end stage renal disease; Raynaud syndrome; and arthritis. In some embodiments, compounds of the invention are administered to a subject in need of treatment of smooth muscle disorders, endothelial disfunction and migraine.

### Methods to Treat Smooth Muscle Disorders

The compounds of this invention are useful to treat smooth muscle disorders, including, but not limited to, erectile dysfunction, overactive bladder, uterine contractions and irritable bowel syndrome.

Smooth muscles can be divided into “multi-unit” and “visceral” types or into “phasic” and “tonic” types based on the characteristics of the contractile patterns. Smooth muscles may contract phasically with rapid contraction and relaxation, or tonically with slow and sustained contraction. The reproductive, digestive, respiratory, and urinary tracts, skin, eye, and vasculature all contain this tonic muscle type. By way of example, contractile and relaxation function of vascular smooth muscle is critical to regulating the lumenal diameter of the small arteries-arterioles called resistance vessels. The resistance arteries contribute significantly to setting the level of blood pressure. Smooth muscle contracts slowly and may maintain the contraction for prolonged periods in blood vessels, bronchioles, and some sphincters. By way of another example, in the digestive tract, non-vascular smooth muscle contracts in a rhythmic peristaltic fashion, rhythmically forcing foodstuffs through the digestive tract as the result of phasic contraction.

A smooth muscle disorder is characterized by an otherwise healthy smooth muscle which over or under responds to stimuli. Said stimuli are capable of inducing smooth muscle contraction or relaxation as described above. Said stimuli includes, but are not limited to, direct stimulation by the autonomic nervous system, chemical, biological or physical stimulation by neighbouring cells and hormones within the medium that surround the muscle.

Erectile dysfunction (ED) or male impotence is characterized by the regular or repeated inability to obtain or maintain an erection. There are several ways that erectile dysfunction is analyzed including, but not limited to: a) obtaining full erections at some times, such as when asleep, when the mind and psychological issues if any are less present, tends to suggest the physical structures are functionally working; b) obtaining erections which are either not rigid or full (lazy erection), or are lost more rapidly than would be expected (often before or during penetration), can be a sign of a failure of the mechanism which keeps blood held in the penis, and may signify an underlying clinical condition; and c) other factors leading to erectile dysfunction are diabetes mellitus (causing neuropathy) or hypogonadism (decreased testosterone levels due to disease affecting the testicles or the pituitary gland).

There are many causes of ED and are usually multifactorial in a single subject, including but not limited to, organic, physiologic, endocrine, and psychogenic factors. One of the physiological causes of erectile dysfunction is the inability of the smooth muscle comprising the penis to relax thereby allowing the infiltration of blood into the penis. Disorders which result in the insufficiency or defective relaxation of the smooth muscle can result in ED.

Diseases associated with ED include, but are not limited to; vascular diseases such as atherosclerosis, peripheral vascular disease, myocardial infarction, arterial hypertension, vascular diseases resulting from radiaon therapy or prostate cancer treatment, blood vessel and nerve trauma; systemic diseases such as diabetes mellitus, scleroderma, renal failure, liver cirrhosis, idiopathic hemochromatosis, cancer treatment, dyslipidemia and hypertension; neurogenic diseases such as, epilepsy, stroke, multiple sclerosis, Guillain-Barré syndrome, Alzheimers disease and trauma; respiratory diseases such as, chronic obstructive pulmonary disease and sleep apnea; hematologic diseases such as sickle cell anemia and leukemias; endocrine conditions such as, hyperthyroidism, hypothyroidism, hypogonadism and diabetes; penile conditions such as, peyronie disease, epispadias and priapism; and psychiatric conditions such as depression, widower syndrome, performance anxiety and posttraumatic stress disorder. Additional states which are associated with ED include nutritional states such as, malnutrition and zinc deficiency; surgical procedures such as, procedures on the brain and spinal cord, retroperitoneal or pelvic lymph node dissection, aortioliac or aortofemoral bypass, abdominal perineal resection, surgical removal of the prostate, proctocolectomy, transurethral resection of the prostate, and cryosurgery of the prostate; and treat with medication such as, antidepressants, antipsychotics, antihypertensives, antiulcer agents, 5-alpha reductase inhibitors and cholesterol-lowering agents.

Overactive bladder (OAB) is defined by the International Continence Society as a urological condition defined by a set of symptoms: urgency, with and without urge incontinence, usually with frequency and nocturia. The etiology of OAB is still unclear, however it is often associated with detrusuor overactivity, a pattern of bladder muscle contraction observed during urodynamic.

Irritable bowel syndrome (IBS) also known as “spastic colon” is a functional bowel disorder characterized by abdominal pair and altered bowel habits in the absence of specific and unique organic pathology. IBS is a clinically defined disease, wherein one set of criteria is that the subject must have recurrent abdominal pain or discomfort at least 3 days per month during the previous 3 months that is associated with 2 or more of the following: relieved by defecation, onset associated with a change in stool frequency and onset associated with a change in stool form or appearance. Additional symptoms included altered stool frequency, altered stool form, altered stool passage (straining and/or urgency), mucorrhea and abdominal bloating or subjective distention.

Uterine Contraction is the tightening and shortening of the smooth muscles comprising the uterus. Irregular contractions, increased frequency or increased contraction strength of the uterus can be associated with the pre-menstrual syndrome (PMS) or during premature or normal labor delivery of a fetus.

Accordingly, in one aspect the invention provides a method for enhancing smooth muscle function by administering to the subject predisposed to a disorder, disease or condition associated therewith an effective amount of a compound of the invention. In a further aspect, the method enhances the smooth muscle relaxation of non-vascular smooth muscle. This non-vascular smooth muscle in some aspects comprises the male or female reproductive tract, bladder or gastrointestinal tract of said subject.

In another aspect, the invention provides a method for treating a smooth muscle disorder in a subject, wherein the smooth muscle disorder is characterized by an otherwise healthy smooth muscle which over or under responds to stimuli by administering to the subject an effective amount of a compound of the invention. In a further aspect the smooth muscle disorder is not hypertension. In yet a further aspect the subject is suffering from a smooth muscle disorder selected from, but not limited to, erectile dysfunction, overactive bladder, uterine contractions, irritable bowel syndrome, non-inflammatory irritable bowel syndrome, migraine, general gastrointestinal tract motility.

In a further aspect of the above embodiments, a subject is unable to be treated with an effective amount of a phosphodiesterase type 5 inhibitor. Examples of phosphodiesterase type 5 inhibitors include, but are not limited to, sildenafil, tadalafil, vardenafil, udenafil and avanafil. In a further aspect, the subject of the above embodiments are unable to be treated with a phosphodiesterase type 5 inhibitor due to a preexisting disease, disorder or condition including, but not limited to, congestive heart failure, heart disease, stroke, hypotension, diabetes or any combination thereof.

In a further aspect of the above embodiments, a subject is unable to be treated with an effective amount of an anticholinergic. Examples of anticholinergics include, but are not limited to, dicycloverine, tolterodine, oxybutynin, trospium and solifenacin.

### Methods to Treat ARDS and SIRS

Adult respiratory distress syndrome (ARDS) is a pulmonary disease that has a mortality rate of 50% and results from lung lesions that are caused by a variety of conditions found in trauma patients and in severe burn victims. Ingram, R. H. Jr., “Adult Respiratory Distress Syndrome,” Harrison's Principals of Internal Medicine, 13, p. 1240, 1995. With the possible exception of glucocorticoids, there have not been therapeutic agents known to be effective in preventing or ameliorating the tissue injury, such as microvascular damage, associated with acute inflammation that occurs during the early development of ARDS.

ARDS, which is defined in part by the development of alveolar edema, represents a clinical manifestation of pulmonary disease resulting from both direct and indirect lung injury. While previous studies have detailed a seemingly unrelated variety of causative agents, the initial events underlying the pathophysiology of ARDS are not well understood. ARDS was originally viewed as a single organ failure, but is now considered a component of the multisystem organ failure syndrome (MOFS). Pharmacologic intervention or prevention of the inflammatory response is presently viewed as a more promising method of controlling the disease process than improved ventilatory support techniques. See, for example, Demling, Annu. Rev. Med., 46, pp. 193-203, 1995.

Another disease (or group of diseases) involving acute inflammation is the systematic inflammatory response syndrome, or SIRS, which is the designation recently established by a group of researchers to describe related conditions resulting from, for example, sepsis, pancreatitis, multiple trauma such as injury to the brain, and tissue injury, such as laceration of the musculature, brain surgery, hemorrhagic shock, and immune-mediated organ injuries (JAMA, 268(24):3452-3455 (1992)).

The ARDS ailments are seen in a variety of patients with severe burns or sepsis. Sepsis in turn is one of the SIRS symptoms. In ARDS, there is an acute inflammatory reaction with high numbers of neutrophils that migrate into the interstitium and alveoli. If this progresses there is increased inflammation, edema, cell proliferation, and the end result is impaired ability to extract oxygen. ARDS is thus a common complication in a wide variety of diseases and trauma. The only treatment is supportive. There are an estimated 150,000 cases per year and mortality ranges from 10% to 90%.

The exact cause of ARDS is not known. However it has been hypothesized that over-activation of neutrophils leads to the release of linoleic acid in high levels via phospholipase A₂ activity. Linoleic acid in turn is converted to 9,10-epoxy-12-octadecenoate enzymatically by neutrophil cytochrome P-450 epoxygenase and/or a burst of active oxygen. This lipid epoxide, or leukotoxin, is found in high levels in burned skin and in the serum and bronchial lavage of burn patients. Furthermore, when injected into rats, mice, dogs, and other mammals it causes ARDS. The mechanism of action is not known. However, the leukotoxin diol produced by the action of the soluble epoxide hydrolase appears to be a specific inducer of the mitochondrial inner membrane permeability transition (MPT). This induction by leukotoxin diol, the diagnostic release of cytochrome c, nuclear condensation, DNA laddering, and CPP32 activation leading to cell death were all inhibited by cyclosporin A, which is diagnostic for MPT induced cell death. Actions at the mitochondrial and cell level were consistent with this mechanism of action suggesting that the inhibitors of this invention could be used therapeutically with compounds which block MPT.

Thus in one embodiment provided is a method for treating ARDS. In another embodiment, provided is a method for treating SIRS.

### Treatment of Endothelial Dysfunction

This invention also provides methods and compositions that treat, reduce or ameliorate the diseases or the symptoms of diseases related to endothelial dysfunction using one or more sEH inhibitors of this invention.

The endothelium is a cellular layer lining the walls of blood vessels of a mammal. It is a highly specialized interface between blood and underlying tissues and has a number of functions, including: control of haemostasis by inhibiting platelet aggregation (antithrombotic and regulating the coagulation and fibrolinolytic systems); control of vascular tone, and hence blood flow; control of blood vessel smooth muscle growth; and selective permeability to cells and proteins.

Normally, the endothelium maintains vascular homeostasis by responding to physiological stimuli, for example, changes in blood flow, oxygen tension etc., by adaptive alteration of function. Dysfunctional endothelium has an impaired response to such physiological stimuli, and can ultimately lead to medical disorders. A number of subsets of endothelial dysfunction have been recognized, including Endothelial Activation, and Endothelial-mediated Vasodilatory Dysfunction (see De Caterina “Endothelial dysfunctions: common denominators in vascular disease”. Current Opinions in Lipidology 11:9-23, (2000)).

Endothelial activation may lead to the initiation of atherosclerosis and is a process whereby there is an inappropriate up-regulation and expression of cell attraction and cell adhesion molecules on endothelial cells. This particularly involves the Macrophage Chemoattractant Protein-1 (MCP-1), chemoattractants for lymphocytes (IP-10, MIG, I-TAG), the Vascular Cell Adhesion Molecule-1 (VCAM-1), IL-1, IL-6, TNFα, and ICAM-1, to which the monocytes and lymphocytes adhere. Once adherent, the leucocytes enter the artery wall. The monocytes and lymphocytes are recruited to the intima (sub-endothelial layers) of the blood vessels by these cell attraction and cell adhesion molecules of the activated endothelium during the early stages of atherosclerosis (see Libby, P. “Changing concepts of atherogenesis,” Journal of Internal Medicine 247:349-358, (2000)).

Endothelial-mediated Vasodilatory Dysfunction is characterized by a reduction or loss of endothelium-dependent vasodilation and involves “decreased nitric oxide bioavailability” (decreased production, increased destruction and/or decreased sensitivity to nitric oxide). (De Caterina (2000), cited above). Nitric oxide induces vasodilation by relaxing the smooth muscle cells of the blood vessel wall. Endothelial-mediated Vasodilatory Dysfunction can be measured as a reduction in vasodilation in response to acetylcholine, or as a reduced vasodilatory response following occlusion of arterial blood flow (reactive hyperaemia) for example using a sphygmomanometer cuff. As well as leading to a reduction in vasodilation, decreased endothelial nitric oxide bioavailability can also result in an increase in the production of vaso-constriction and hypertension. Platelet aggregation is inhibited by nitric oxide, hence a decrease in nitric oxide bioavailability can lead to an increase in platelet aggregation and consequent thrombosis. These are just a few examples of how decreased nitric oxide bioavailability resulting from Endothelial-mediated Vasodilatory Dysfunction can have pathological consequences.

A variety of diseases related to endothelial dysfunction that can be treated in the present invention, include, by way of example only, vascular inflammation, such as, atherosclerosis plaque progression/rupture and acute coronary syndrome; vasospasm, such as, coronary-angina and cerebral-subarachnoid hemorrhage; nephropathy, such as, micro-albuminuria; diabetic vasculopathy; and autoimmune vasculitis. In one embodiment, the autoimmune vasculitis relates to scleroderma, lupus, behcet syndrome, takayashu arteritis, churg-strauss syndrome, cutaneous vasculitis, and thrombangitis obliterans (Raynaud's syndrome). In one embodiment, autoimmune vasculitis is associated with sickle cell anemia and beta thalasemia.

Sickle cell anemia is characterized by several aspects that make it a disease that may be positively impacted by inhibition of sEH. Although the anemia is congenital, the acute sickling events lead to the actual issues with the disease including vascular inflammation, stroke and renal damage. Vascular inflammation may be considered a key characteristic of this disease. Stroke is a co-morbidity in sickle cell anemia that has potential to be positively impacted by sEH inhibitors. Additionally, it is also characterized by leading to a wide range of glomerular and tubulointerstitial nephropathies. Finally, an sEH inhibitor can be anti-thrombotic which can positively impact the primary mortality.

In one embodiment, the invention provides methods and compositions that treat, reduce or ameliorate the diseases or the symptoms of diseases related to vascular inflammation, using one or more compound(s) of this invention.

Functional tests/diagnosis normally used to screen for diseases related to endothelial dysfunction include but are not limited to, flow-mediated arterial dilation (FMAD) usually measured non-invasively in the patients' forearm (brachial artery) and measurement of acetylcholine-induced arterial dilation. The biochemical markers measured in patients blood/plasma include but are not limited to, soluble Vascular Cell Adhesion Molecule-1 (VCAM-1), Intercellular Adhesion Molecule-1 (ICAM-1), Platelet/endothelial Cell Adhesion Molecule-1 (PECAM-1) and von Willebrand Factor (vWF). Functional tests/diagnosis normally used to screen for diseases related to vascular inflammation include, but are not limited to, blood/plasma markers such as above and/or TNFα, IL-1, IL-6, MCP-1, NOx, etc. and clinical symptoms.

One can determine if the treatment has been effective for its defined purpose by noting one or more clinical symptoms such as a reduction in pain, redness, swelling and loss of mobility or function. Administration of compositions of the invention can be further selected on their ability to reduce clinical symptoms by at least 50%, or alternatively, at least by about 60% or alternatively by at least about 70%, or alternatively by at least about 75%, or alternatively by at least about 80%, or alternatively by at least about 85%, or alternatively by at least about 90%, or alternatively by at least about 95%, of pre-administration levels in the subject.

Also provided is a medicament comprising one or more compound(s) of the invention for use in treating a disease or disorder as described in the methods above, which can be identified by noting any one or more clinical or sub-clinical parameters.

### Methods for Inhibiting Progression of Kidney Deterioration (Nephropathy) and Reducing Blood Pressure:

In another aspect of the invention, the compounds of the invention can reduce damage to the kidney, and especially damage to kidneys from diabetes, as measured by albuminuria. It is contemplated that the compounds of the invention can reduce kidney deterioration (nephropathy) from diabetes even in individuals who do not have high blood pressure. The conditions of therapeutic administration are as described above.

cis-Epoxyeicosantrienoic acids (“EETs”) can be used in conjunction with the compounds of the invention to further reduce kidney damage. EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into DHETs is reduced. Without wishing to be bound by theory, it is believed that raising the level of EETs interferes with damage to kidney cells by the microvasculature changes and other pathologic effects of diabetic hyperglycemia. Therefore, raising the EET level in the kidney is believed to protect the kidney from progression from microalbuminuria to end stage renal disease.

EETs are well known in the art. EETs useful in the methods of the present invention include 14,15-EET, 8,9-EET and 11,12-EET, and 5,6 EETs, in that order of preference. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

EETs produced by the endothelium have anti-hypertensive properties and the EETs 11,12-EET and 14,15-EET may be endothelium-derived hyperpolarizing factors (EDHFs). Additionally, EETs such as 11,12-EET have profibrinolytic effects, anti-inflammatory actions and inhibit smooth muscle cell proliferation and migration. In the context of the present invention, these favorable properties are believed to protect the vasculature and organs during renal and cardiovascular disease states.

Inhibition of sEH activity can be effected by increasing the levels of EETs. This permits EETs to be used in conjunction with one or more sEH inhibitors to reduce nephropathy in the methods of the invention. It further permits EETs to be used in conjunction with one or more sEH inhibitors to reduce hypertension, or inflammation, or both. Thus, medicaments of EETs can be made which can be administered in conjunction with one or more sEH inhibitors, or a medicament containing one or more sEH inhibitors can optionally contain one or more EETs.

The EETs can be administered concurrently with the sEH inhibitor, or following administration of the sEH inhibitor. It is understood that, like all drugs, inhibitors have half lives defined by the rate at which they are metabolized by or excreted from the body, and that the inhibitor will have a period following administration during which it will be present in amounts sufficient to be effective. If EETs are administered after the inhibitor is administered, therefore, it is desirable that the EETs be administered during the period in which the inhibitor will be present in amounts to be effective to delay hydrolysis of the EETs. Typically, the EET or EETs will be administered within 48 hours of administering an sEH inhibitor. Preferably, the EET or EETs are administered within 24 hours of the inhibitor, and even more preferably within 12 hours. In increasing order of desirability, the EET or EETs are administered within 10, 8, 6, 4, 2, hours, 1 hour, or one half hour after administration of the inhibitor. Most preferably, the EET or EETs are administered concurrently with the inhibitor.

In preferred embodiments, the EETs, the compound of the invention, or both, are provided in a material that permits them to be released over time to provide a longer duration of action. Slow release coatings are well known in the pharmaceutical art; the choice of the particular slow release coating is not critical to the practice of the present invention.

EETs are subject to degradation under acidic conditions. Thus, if the EETs are to be administered orally, it is desirable that they are protected from degradation in the stomach. Conveniently, EETs for oral administration may be coated to permit them to passage through the acidic environment of the stomach into the basic environment of the intestines. Such coatings are well known in the art. For example, aspirin coated with so-called “enteric coatings” is widely available commercially. Such enteric coatings may be used to protect EETs during passage through the stomach. An exemplary coating is set forth in the Examples.

Further, administration of exogenous EETs in conjunction with an sEH inhibitor is expected to be beneficial and to augment the effects of the sEH inhibitor in reducing the progression of diabetic nephropathy.

The present invention can be used in regard to any and all forms of diabetes to the extent that they are associated with progressive damage to the kidney or kidney function. The chronic hyperglycemia of diabetes is associated with long-term damage, dysfunction, and failure of various organs, especially the eyes, kidneys, nerves, heart, and blood vessels. The long-term complications of diabetes include retinopathy with potential loss of vision; nephropathy leading to renal failure; peripheral neuropathy with risk of foot ulcers, amputation, and Charcot joints.

In addition, persons with metabolic syndrome are at high risk of progression to type 2 diabetes, and therefore at higher risk than average for diabetic nephropathy. It is therefore desirable to monitor such individuals for microalbuminuria, and to administer an sEH inhibitor and, optionally, one or more EETs, as an intervention to reduce the development of nephropathy. The practitioner may wait until microalbuminuria is seen before beginning the intervention. Since a person can be diagnosed with metabolic syndrome without having a blood pressure of 130/85 or higher, both persons with blood pressure of 130/85 or higher and persons with blood pressure below 130/85 can benefit from the administration of sEH inhibitors and, optionally, of one or more EETs, to slow the progression of damage to their kidneys. In some preferred embodiments, the person has metabolic syndrome and blood pressure below 130/85.

Dyslipidemia or disorders of lipid metabolism is another risk factor for heart disease. Such disorders include an increased level of LDL cholesterol, a reduced level of HDL cholesterol, and an increased level of triglycerides. An increased level of serum cholesterol, and especially of LDL cholesterol, is associated with an increased risk of heart disease. The kidneys are also damaged by such high levels. It is believed that high levels of triglycerides are associated with kidney damage. In particular, levels of cholesterol over 200 mg/dL, and especially levels over 225 mg/dL, would suggest that sEH inhibitors and, optionally, EETs, should be administered. Similarly, triglyceride levels of more than 215 mg/dL, and especially of 250 mg/dL or higher, would indicate that administration of sEH inhibitors and, optionally, of EETs, would be desirable. The administration of compounds of the present invention with or without the EETs, can reduce the need to administer statin drugs (HMG-COA reductase inhibitors) to the patients, or reduce the amount of the statins needed. In some embodiments, candidates for the methods, uses, and compositions of the invention have triglyceride levels over 215 mg/dL and blood pressure below 130/85. In some embodiments, the candidates have triglyceride levels over 250 mg/dL and blood pressure below 130/85. In some embodiments, candidates for the methods, uses and compositions of the invention have cholesterol levels over 200 mg/dL and blood pressure below 130/85. In some embodiments, the candidates have cholesterol levels over 225 mg/dL and blood pressure below 130/85.

### Methods of Inhibiting the Proliferation of Vascular Smooth Muscle Cells:

In other embodiments, compounds of any one of Formulas (I), (IIa), (IIb), (IIIa), (IIIb), (IVa), (IVb), and (IVc), or of Table 1 inhibit proliferation of vascular smooth muscle (VSM) cells without significant cell toxicity, (e.g. specific to VSM cells). Because VSM cell proliferation is an integral process in the pathophysiology of atherosclerosis, these compounds are suitable for slowing or inhibiting atherosclerosis. These compounds are useful to subjects at risk for atherosclerosis, such as individuals who have diabetes and those who have had a heart attack or a test result showing decreased blood circulation to the heart. The conditions of therapeutic administration are as described above.

The methods of the invention are particularly useful for patients who have had percutaneous intervention, such as angioplasty to reopen a narrowed artery, to reduce or to slow the narrowing of the reopened passage by restenosis. In some preferred embodiments, the artery is a coronary artery. The compounds of the invention can be placed on stents in polymeric coatings to provide a controlled localized release to reduce restenosis. Polymer compositions for implantable medical devices, such as stents, and methods for embedding agents in the polymer for controlled release, are known in the art and taught, for example, in U.S. Pat. Nos. 6,335,029; 6,322,847; 6,299,604; 6,290,722; 6,287,285; and 5,637,113. In preferred embodiments, the coating releases the inhibitor over a period of time, preferably over a period of days, weeks, or months. The particular polymer or other coating chosen is not a critical part of the present invention.

The methods of the invention are useful for slowing or inhibiting the stenosis or restenosis of natural and synthetic vascular grafts. As noted above in connection with stents, desirably, the synthetic vascular graft comprises a material which releases a compound of the invention over time to slow or inhibit VSM proliferation and the consequent stenosis of the graft. Hemodialysis grafts are a particularly preferred embodiment.

In addition to these uses, the methods of the invention can be used to slow or to inhibit stenosis or restenosis of blood vessels of persons who have had a heart attack, or whose test results indicate that they are at risk of a heart attack.

Removal of a clot such as by angioplasty or treatment with tissue plasminogen activator (tPA) can also lead to reperfusion injury, in which the resupply of blood and oxygen to hypoxic cells causes oxidative damage and triggers inflammatory events. In some embodiments, provided are methods for administering the compounds and compositions of the invention for treating reperfusion injury. In some such embodiments, the compounds and compositions are administered prior to or following angioplasty or administration of tPA.

In one group of preferred embodiments, compounds of the invention are administered to reduce proliferation of VSM cells in persons who do not have hypertension. In another group of embodiments, compounds of the invention are used to reduce proliferation of VSM cells in persons who are being treated for hypertension, but with an agent that is not an sEH inhibitor.

The compounds of the invention can be used to interfere with the proliferation of cells which exhibit inappropriate cell cycle regulation. In one important set of embodiments, the cells are cells of a cancer. The proliferation of such cells can be slowed or inhibited by contacting the cells with a compound of the invention. The determination of whether a particular compound of the invention can slow or inhibit the proliferation of cells of any particular type of cancer can be determined using assays routine in the art.

In addition to the use of the compounds of the invention, the levels of EETs can be raised by adding EETs. VSM cells contacted with both an EET and a compound of the invention exhibited slower proliferation than cells exposed to either the EET alone or to the compound of the invention alone. Accordingly, if desired, the slowing or inhibition of VSM cells of a compound of the invention can be enhanced by adding an EET along with a compound of the invention. In the case of stents or vascular grafts, for example, this can conveniently be accomplished by embedding the EET in a coating along with a compound of the invention so that both are released once the stent or graft is in position.

### Methods of Inhibiting the Progression of Obstructive Pulmonary Disease, Interstitial Lung Disease, or Asthma:

Chronic obstructive pulmonary disease, or COPD, encompasses two conditions, emphysema and chronic bronchitis, which relate to damage caused to the lung by air pollution, chronic exposure to chemicals, and tobacco smoke. Emphysema as a disease relates to damage to the alveoli of the lung, which results in loss of the separation between alveoli and a consequent reduction in the overall surface area available for gas exchange. Chronic bronchitis relates to irritation of the bronchioles, resulting in excess production of mucin, and the consequent blocking by mucin of the airways leading to the alveoli. While persons with emphysema do not necessarily have chronic bronchitis or vice versa, it is common for persons with one of the conditions to also have the other, as well as other lung disorders.

Some of the damage to the lungs due to COPD, emphysema, chronic bronchitis, and other obstructive lung disorders may be inhibited or reversed by administering sEH inhibitors of the invention. The effects of sEH inhibitors can be increased by also administering EETs. The effect is at least additive over administering the two agents separately, and may indeed be synergistic.

The studies reported herein show that EETs can be used in conjunction with sEH inhibitors to reduce damage to the lungs by tobacco smoke or, by extension, by occupational or environmental irritants. These findings indicate that the co-administration of sEH inhibitors and of EETs can be used to inhibit or slow the development or progression of COPD, emphysema, chronic bronchitis, or other chronic obstructive lung diseases which cause irritation to the lungs.

Animal models of COPD and humans with COPD have elevated levels of immunomodulatory lymphocytes and neutrophils. Neutrophils release agents that cause tissue damage and, if not regulated, will over time have a destructive effect. Without wishing to be bound by theory, it is believed that reducing levels of neutrophils reduces tissue damage contributing to obstructive lung diseases such as COPD, emphysema, and chronic bronchitis. Administration of sEH inhibitors to rats in an animal model of COPD resulted in a reduction in the number of neutrophils found in the lungs. Administration of EETs in addition to the sEH inhibitors is expected to produce a greater reduction in neutrophil levels than in the presence of the sEH inhibitor alone.

This is particularly advantageous where the diseases or other factors have reduced the endogenous concentrations of EETs below those normally present in healthy individuals. Administration of exogenous EETs in conjunction with an sEH inhibitor is therefore expected to augment the effects of the sEH inhibitor in inhibiting or reducing the progression of COPD or other pulmonary diseases.

In addition to inhibiting or reducing the progression of chronic obstructive airway conditions, the invention also provides new ways of reducing the severity or progression of chronic restrictive airway diseases. While obstructive airway diseases tend to result from the destruction of the lung parenchyma, and especially of the alveoli, restrictive diseases tend to arise from the deposition of excess collagen in the parenchyma. These restrictive diseases are commonly referred to as “interstitial lung diseases”, or “ILDs”, and include conditions such as idiopathic pulmonary fibrosis. The compounds and compositions of the invention are useful for reducing the severity or progression of ILDs, such as idiopathic pulmonary fibrosis. Macrophages play a significant role in stimulating interstitial cells, particularly fibroblasts, to lay down collagen. Without wishing to be bound by theory, it is believed that neutrophils are involved in activating macrophages.

In some preferred embodiments, the ILD is idiopathic pulmonary fibrosis. In other preferred embodiments, the ILD is one associated with an occupational or environmental exposure. Exemplars of such ILDs, are asbestosis, silicosis, coal worker's pneumoconiosis, and berylliosis. Further, occupational exposure to any of a number of inorganic dusts and organic dusts is believed to be associated with mucus hypersecretion and respiratory disease, including cement dust, coke oven emissions, mica, rock dusts, cotton dust, and grain dust (for a more complete list of occupational dusts associated with these conditions, see Table 254-1 of Speizer, “Environmental Lung Diseases,” Harrison's Principles of Internal Medicine, infra, at pp. 1429-1436). In other embodiments, the ILD is sarcoidosis of the lungs. ILDs can also result from radiation in medical treatment, particularly for breast cancer, and from connective tissue or collagen diseases such as rheumatoid arthritis and systemic sclerosis. It is believed that the compounds and compositions of the invention can be useful in each of these interstitial lung diseases.

In another set of embodiments, the invention is used to reduce the severity or progression of asthma. Asthma typically results in mucin hypersecretion, resulting in partial airway obstruction. Additionally, irritation of the airway results in the release of mediators which result in airway obstruction. While the lymphocytes and other immunomodulatory cells recruited to the lungs in asthma may differ from those recruited as a result of COPD or an ILD, it is expected that the invention will reduce the influx of immunomodulatory cells, such as neutrophils and eosinophils, and ameliorate the extent of obstruction. Thus, it is expected that the administration of sEH inhibitors, and the administration of sEH inhibitors in combination with EETs, will be useful in reducing airway obstruction due to asthma.

In each of these diseases and conditions, it is believed that at least some of the damage to the lungs is due to agents released by neutrophils which infiltrate into the lungs. The presence of neutrophils in the airways is thus indicative of continuing damage from the disease or condition, while a reduction in the number of neutrophils is indicative of reduced damage or disease progression. Thus, a reduction in the number of neutrophils in the airways in the presence of an agent is a marker that the agent is reducing damage due to the disease or condition, and is slowing the further development of the disease or condition. The number of neutrophils present in the lungs can be determined by, for example, bronchoalveolar lavage.

### Prophylactic and Therapeutic Methods to Reduce Stroke Damage:

Inhibitors of sEH and EETs administered in conjunction with inhibitors of sEH have been shown to reduce brain damage from strokes. Based on these results, we expect that inhibitors of sEH taken prior to an ischemic stroke will reduce the area of brain damage and will likely reduce the consequent degree of impairment. The reduced area of damage should also be associated with a faster recovery from the effects of the stroke.

While the pathophysiologies of different subtypes of stroke differ, they all cause brain damage. Hemorrhagic stroke differs from ischemic stroke in that the damage is largely due to compression of tissue as blood builds up in the confined space within the skull after a blood vessel ruptures, whereas in ischemic stroke, the damage is largely due to loss of oxygen supply to tissues downstream of the blockage of a blood vessel by a clot. Ischemic strokes are divided into thrombotic strokes, in which a clot blocks a blood vessel in the brain, and embolic strokes, in which a clot formed elsewhere in the body is carried through the blood stream and blocks a vessel there. In both hemorrhagic stroke and ischemic stroke, the damage is due to the death of brain cells. Based on the results observed in our studies, we would expect at least some reduction in brain damage in all types of stroke and in all subtypes.

A number of factors are associated with an increased risk of stroke. Given the results of the studies underlying the present invention, sEH inhibitors administered to persons with any one or more of the following conditions or risk factors: high blood pressure, tobacco use, diabetes, carotid artery disease, peripheral artery disease, atrial fibrillation, transient ischemic attacks (TIAs), blood disorders such as high red blood cell counts and sickle cell disease, high blood cholesterol, obesity, alcohol use of more than one drink a day for women or two drinks a day for men, use of cocaine, a family history of stroke, a previous stroke or heart attack, or being elderly, will reduce the area of brain damaged by a stroke. With respect to being elderly, the risk of stroke increases for every 10 years. Thus, as an individual reaches 60, 70, or 80, administration of sEH inhibitors has an increasingly larger potential benefit. As noted in the next section, the administration of EETs in combination with one or more sEH inhibitors can be beneficial in further reducing the brain damage.

In some preferred uses and methods, the sEH inhibitors and, optionally, EETs, are administered to persons who use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

Clot dissolving agents, such as tissue plasminogen activator (tPA), have been shown to reduce the extent of damage from ischemic strokes if administered in the hours shortly after a stroke. For example, tPA is approved by the FDA for use in the first three hours after a stroke. Thus, at least some of the brain damage from a stoke is not instantaneous, but rather occurs over a period of time or after a period of time has elapsed after the stroke. It is contemplated that administration of sEH inhibitors, optionally with EETs, can also reduce brain damage if administered within 6 hours after a stroke has occurred, more preferably within 5, 4, 3, or 2 hours after a stroke has occurred, with each successive shorter interval being more preferable. Even more preferably, the inhibitor or inhibitors are administered 2 hours or less or even 1 hour or less after the stroke, to maximize the reduction in brain damage. Persons of skill are well aware of how to make a diagnosis of whether or not a patient has had a stroke. Such determinations are typically made in hospital emergency rooms, following standard differential diagnosis protocols and imaging procedures.

In some preferred uses and methods, the sEH inhibitors and, optionally, EETs, are administered to persons who have had a stroke within the last 6 hours who: use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

### Metabolic Syndrome

Inhibitors of soluble epoxide hydrolase (“sEH”) and EETs administered in conjunction with inhibitors of sEH have been shown to treat one or more conditions associated with metabolic syndrome as provided for in U.S. Patent Application Publication Nos. 2008/0221105, and U.S. patent application Ser. No. 12/264,816, which are incorporated herein by reference in their entirety.

Metabolic syndrome is characterized by a group of metabolic risk factors present in one person. The metabolic risk factors include central obesity (excessive fat tissue in and around the abdomen), atherogenic dyslipidemia (blood fat disorders—mainly high triglycerides and low HDL cholesterol), insulin resistance or glucose intolerance, prothrombotic state (e.g., high fibrinogen or plasminogen activator inhibitor in the blood), and high blood pressure (130/85 mm Hg or higher).

Metabolic syndrome, in general, can be diagnosed based on the presence of three or more of the following clinical manifestations in one subject:

a) Abdominal obesity characterized by a elevated waist circumference equal to or greater than 40 inches (102 cm) in men and equal to or greater than 35 inches (88 cm) in women or obesity characterized by a body mass index (BMI) equal to or greater than 25, or in another aspect a BMI equal to or greater than 30, or in another aspect a BMI equal to or greater than 35, or in yet another aspect a BMI equal to or greater than 40;

b) Elevated triglycerides equal to or greater than 150 mg/dL or in one aspect equal to or greater than 200 mg/dL, or in another aspect less than 215 mg/dL, or in another aspect equal to or greater than 150 mg/dL but less than 200 mg/dL, or in yet another aspect equal to or greater than 150 mg/dL but less than 215 mg/dL;

c) Reduced levels of high-density lipoproteins of less than 40 mg/dL in women and less than 50 mg/dL in men, or alternatively less than 35 mg/dL in women and less than 45 mg/dL in men, or alternatively less than 30 mg/dL in women and less than 40 mg/dL in men, or alternatively between 10 mg/dL to 40 mg/dL in women and between 10 mg/dL to 50 mg/dL in men, or alternatively between 15 mg/dL to 40 mg/dL in women and between 15 mg/dL to 50 mg/dL in men, or alternatively, between 20 mg/dL to 40 mg/dL in women and between 20 mg/dL to 50 mg/dL in men, or alternatively between 40 mg/dL to 50 mg/dL for both men and women;

d) High blood pressure equal to or greater than 130/85 mm Hg or alternatively equal to or greater than 140/90, or alternatively equal to or greater than 150/90, or alternatively equal to or greater than 140/100, or alternatively equal to or greater than 150/100; and

e) Elevated fasting glucose equal to or greater than 100 mg/dL Elevated fasting glucose equal to or greater than 100 mg/dL, or alternatively, equal to or greater than 110 mg/dL, or alternatively equal to or greater than 120, or alternatively equal to or greater than 100 mg/dL, but in all cases less than 125 mg/dL.

Another risk factor includes reduced ratios of high-density lipoprotein (HDL) to low-density lipoprotein (LDL) of less than 0.4, or alternatively less than 0.3, or alternatively less than 0.2, or alternatively less than 0.1, or alternatively less than 0.4 but equal to or greater than 0.3, or alternatively less than 0.3 but equal to or greater than 0.2 or alternatively less than 0.2 but equal to or greater than 0.1.

It is desirable to provide early intervention to prevent the onset of metabolic syndrome so as to avoid the medical complications brought on by this syndrome. Prevention or inhibition of metabolic syndrome refers to early intervention in subjects predisposed to, but not yet manifesting, metabolic syndrome. These subjects may have a genetic disposition associated with metabolic syndrome and/or they may have certain external acquired factors associated with metabolic syndrome, such as excess body fat, poor diet, or physical inactivity. Additionally, these subjects may exhibit one or more of the conditions associated with metabolic syndrome. These conditions can be in their incipient form. It is contemplated that compounds of this invention are useful in various aspects of treating or inhibiting metabolic syndrome or a condition associated therewith.

Accordingly, in one aspect, the invention provides a method for inhibiting the onset of metabolic syndrome by administering to the subject predisposed thereto an effective amount of a sEH inhibitor of the invention.

Another aspect provides a method for treating one or more conditions associated with metabolic syndrome in a subject where the conditions are selected from incipient diabetes, obesity, glucose intolerance, high blood pressure, elevated serum cholesterol, a reduced ratio of high-density lipoproteins to low-density lipoproteins and elevated triglycerides. This method comprises administering to the subject an amount of an sEH inhibitor effective to treat the condition or conditions manifested in the subject. In one embodiment of this aspect, two or more of the noted conditions are treated by administering to the subject an effective amount of an sEH inhibitor. In this aspect, the conditions to be treated include treatment of hypertension.

sEH inhibitors are also useful in treating metabolic conditions comprising obesity, glucose intolerance, hypertension, high blood pressure, elevated levels of serum cholesterol, a reduced ratio of high-density lipoproteins to low-density lipoproteins and elevated levels of triglycerides, or combinations thereof, regardless if the subject is manifesting, or is predisposed to, metabolic syndrome.

Accordingly, another aspect of the invention provides for methods for treating a metabolic condition in a subject, comprising administering to the subject an effective amount of a sEH inhibitor, wherein the metabolic condition is selected from the group consisting of conditions comprising obesity, glucose intolerance, high blood pressure, elevated serum cholesterol, a reduced ratio of high-density lipoproteins to low-density lipoproteins and elevated triglycerides, and combinations thereof.

In general, levels of glucose, serum cholesterol, triglycerides, obesity, and blood pressure are well known parameters and are readily determined using methods known in the art.

Several distinct categories of glucose intolerance exist, including for example, type 1 diabetes mellitus, type 2 diabetes mellitus, gestational diabetes mellitus (GDM), impaired glucose tolerance (IGT), and impaired fasting glucose (IFG). IGT and IFG are transitional states from a state of normal glycemia to diabetes. IGT is defined as two-hour glucose levels of 140 to 199 mg per dL (7.8 to 11.0 mmol) on the 75-g oral glucose tolerance test (OGTT), and IFG is defined as fasting plasma glucose (FG) values of 100 to 125 mg per dL (5.6 to 6.9 mmol per L) in fasting patients. These glucose levels are above normal but below the level that is diagnostic for diabetes. Rao, et al., Amer. Fam. Phys. 69:1961-1968 (2004).

Current knowledge suggests that development of glucose intolerance or diabetes is initiated by insulin resistance and is worsened by the compensatory hyperinsulinemia. The progression to type 2 diabetes is influenced by genetics and environmental or acquired factors including, for example, a sedentary lifestyle and poor dietary habits that promote obesity. Patients with type 2 diabetes are usually obese, and obesity is also associated with insulin resistance.

“Incipient diabetes” refers to a state where a subject has elevated levels of glucose or, alternatively, elevated levels of glycosylated hemoglobin, but has not developed diabetes. A standard measure of the long term severity and progression of diabetes in a patient is the concentration of glycosylated proteins, typically glycosylated hemoglobin. Glycosylated proteins are formed by the spontaneous reaction of glucose with a free amino group, typically the N-terminal amino group, of a protein. HbA1c is one specific type of glycosylated hemoglobin (Hb), constituting approximately 80% of all glycosylated hemoglobin, in which the N-terminal amino group of the Hb A beta chain is glycosylated.

Formation of HbA1c irreversible and the blood level depends on both the life span of the red blood cells (average 120 days) and the blood glucose concentration. A buildup of glycosylated hemoglobin within the red cell reflects the average level of glucose to which the cell has been exposed during its life cycle. Thus the amount of glycosylated hemoglobin can be indicative of the effectiveness of therapy by monitoring long-term serum glucose regulation. The HbA1c level is proportional to average blood glucose concentration over the previous four weeks to three months. Therefore HbA1c represents the time-averaged blood glucose values, and is not subject to the wide fluctuations observed in blood glucose values, a measurement most typically taken in conjunction with clinical trials of candidate drugs for controlling diabetes.

Obesity can be monitored by measuring the weight of a subject or by measuring the Body Mass Index (BMI) of a subject. BMI is determined by dividing the subject's weight in kilograms by the square of his/her height in metres (BMI=kg/m2). Alternatively, obesity can be monitored by measuring percent body fat. Percent body fat can be measured by methods known in the art including by weighing a subject underwater, by a skinfold test, in which a pinch of skin is precisely measured to determine the thickness of the subcutaneous fat layer, or by bioelectrical impedance analysis.

### Combination Therapy

As noted above, the compounds of the present invention will, in some instances, be used in combination with other therapeutic agents to bring about a desired effect. Selection of additional agents will, in large part, depend on the desired target therapy (see, e.g., Turner, N. et al. Prog. Drug Res. (1998) 51: 33-94; Haffner, S. Diabetes Care (1998) 21: 160-178; and DeFronzo, R. et al. (eds), Diabetes Reviews (1997) Vol. 5 No. 4). A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W., (ed), Current Therapy In Endocrinology And Metabolism, 6th Edition (Mosby-Year Book, Inc., St. Louis, Mo. 1997); Chiasson, J. et al., Ann. Intern. Med. (1994) 121: 928-935; Coniff, R. et al., Clin. Ther. (1997) 19:16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). Combination therapy includes administration of a single pharmaceutical dosage formulation which contains a compound of any one of Formulas (I), (IIa), (IIb), (IIIa), (IIIb), (IVa), (IVb), and (IVc), or of Table 1 and one or more additional active agents, as well as administration of the compound and each active agent in its own separate pharmaceutical dosage formulation. For example, a compound of any one of Formulas (I), (IIa), (IIb), (IIIa), (IIIb), (IVa), (IVb), and (IVc), or of Table 1 and one or more angiotensin receptor blockers, angiotensin converting enzyme inhibitors, calcium channel blockers, diuretics, alpha blockers, beta blockers, centrally acting agents, vasopeptidase inhibitors, renin inhibitors, endothelin receptor agonists, AGE (advanced glycation end-products) crosslink breakers, sodium/potassium ATPase inhibitors, endothelin receptor agonists, endothelin receptor antagonists, angiotensin vaccine, and the like; can be administered to the human subject together in a single oral dosage composition, such as a tablet or capsule, or each agent can be administered in separate oral dosage formulations. Where separate dosage formulations are used, the compound of any one of Formulas (I), (IIa), (IIb), (IIIa), (IIIb), (IVa), (IVb), and (IVc), or of Table 1 and one or more additional active agents can be administered at essentially the same time (i.e., concurrently), or at separately staggered times (i.e., sequentially). Combination therapy is understood to include all these regimens.

### Administration and Pharmaceutical Compositions

In general, the compounds of this invention will be administered in a therapeutically effective amount by any of the accepted modes of administration for agents that serve similar utilities. Therapeutically effective amount is an amount of one or more of the compounds described herein which treats a soluble epoxide hydrolase mediated disease. It is contemplated that a therapeutically effective amount of one or more of the compounds described herein will inhibit the activity of soluble epoxide hydrolase in a patient as compared to the activity of soluble epoxide hydrolase in the absence of treatment. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

Generally, therapeutically effective amounts of the compounds may range from approximately 0.05 to 50 mg per kilogram body weight of the recipient per day; preferably about 0.1-25 mg/kg/day, more preferably from about 0.5 to 10 mg/kg/day. Thus, for administration to a 70 kg person, the dosage range would preferably be about 3.5-2000 mg per day.

In general, compounds of this invention will be administered as pharmaceutical compositions by any one of the following routes: oral, systemic (e.g., transdermal, intranasal or by suppository), parenteral (e.g., intramuscular, intravenous or subcutaneous), or intrathecal administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the invention in combination with at least one pharmaceutically acceptable excipient. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art. Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of the compound of based on the total formulation, with the balance being one or more suitable pharmaceutical excipients. Preferably, the compound is present at a level of about 1-80 wt %. Representative pharmaceutical formulations containing a compound of any one of Formulas (I), (IIa), (IIb), (IIIa), (IIIb), (IVa), (IVb), and (IVc), or of Table 1 are described below.

### General Synthetic Methods

The compounds of this invention can be prepared from readily available starting materials using synthetic methods known in the art, such as the following general methods and procedures. It will be appreciated that where typical or preferred process conditions (i.e., reaction temperatures, times, mole ratios of reactants, solvents, pressures, etc) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

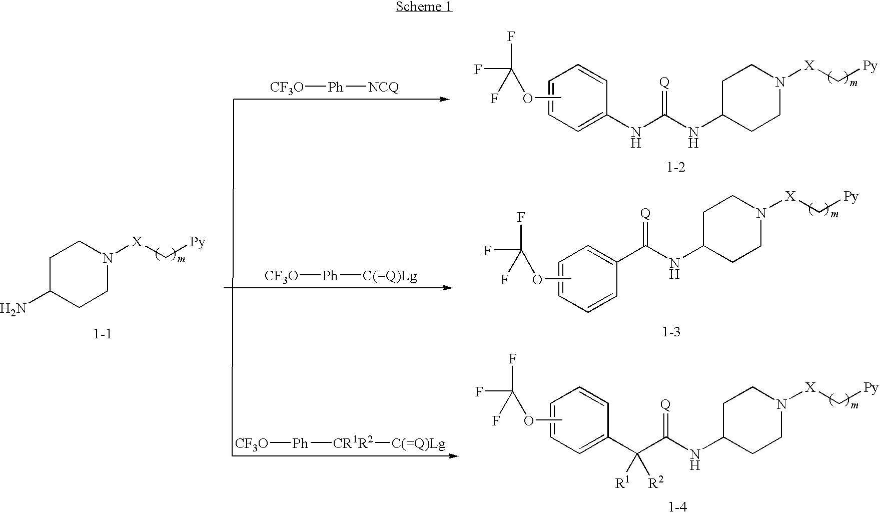

A synthesis of the compounds of the invention is shown in Scheme 1, where R¹, R², Q, X, m, and Py are previously defined, and where Lg is OH or a leaving group, such as halogen. Amine 1-1 can be used as a starting material to form a variety of compounds having a urea, thiourea, amide or thioamide linkage.

Reaction of 1-1 with trifluorophenylisocyanate or trifluorophenylisothiocyanate gives the corresponding urea or thiourea 1-2. Typically, the preparation of the urea is conducted using a polar solvent such as DMF (dimethylformamide) at 60 to 85° C.

Compound 1-1 can react with CF₃OPhC(=Q)Lg or CF₃OPhCR¹R²C(=Q)Lg, where Lg is a leaving group or OH, under amide forming conditions to give the amides 1-3 and 1-4, respectively.

When Lg is OH, a variety of amide coupling reagents may be used to from the amide bond, including the use of carbodiimides such as N—N′-dicyclohexylcarbodiimide (DCC), N—N′-diisopropylcarbodiimide (DIPCDI), and 1-ethyl-3-(3′-dimethylaminopropyl)carbodiimide (EDCI). The carbodiimides may be used in conjunction with additives such as dimethylaminopyridine (DMAP) or benzotriazoles such as 7-aza-1-hydroxybenzotriazole (HOAt), 1-hydroxybenzotriazole (HOBt), and 6-chloro-1-hydroxybenzotriazole (Cl—HOBt).

Amide coupling reagents also include amininum and phosphonium based reagents. Aminium salts include N-[(dimethylamino)-1H-1,2,3-triazolo[4,5-b]pyridine-1-ylmethylene]-N-methylmethanaminium hexafluorophosphate N-oxide (HATU), N-[(1H-benzotriazol-1-yl)(dimethylamino)methylene]-N-methylmethanaminium hexafluorophosphate N-oxide (HBTU), N-[(1H-6-chlorobenzotriazol-1-yl)(dimethylamino)methylene]-N-methylmethanaminium hexafluorophosphate N-oxide (HCTU), N-[(1H-benzotriazol-1-yl)(dimethylamino)methylene]-N-methylmethanaminium tetrafluoroborate N-oxide (TBTU), and N-[(1H-6-chlorobenzotriazol-1-yl)(dimethylamino)methylene]-N-methylmethanaminium tetrafluoroborate N-oxide (TCTU). Phosphonium salts include 7-azabenzotriazol-1-yl-N-oxy-tris(pyrrolidino)phosphonium hexafluorophosphate (PyAOP) and benzotriazol-1-yl-N-oxy-tris(pyrrolidino)phosphonium hexafluorophosphate (PyBOP). Amide formation step may be conducted in a polar solvent such as dimethylformamide (DMF) and may also include an organic base such as diisopropylethylamine (DIEA) or dimethylaminopyridine (DMAP).

Generally, amine 1-1 may be readily available from commercial sources or prepared by conventional methods and procedures known to a person of skill in the art.

Alternatively, compounds of this invention may be prepared according to Scheme 2 from compounds of Formula 2-1

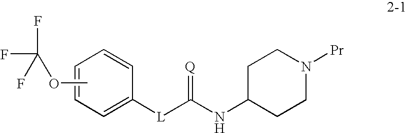

wherein L is as defined herein and Pr is an amino protecting group, such as tert-butoxycarbonyl (Boc), benzyloxycarbonyl (Cbz), and 9-fluorenylmethyloxycarbonyl (Fmoc). Compounds of Formula 2-1 may be prepared using a method similar to Scheme 1 for the preparation of Formulas 1-2, 1-3 or 1-4.

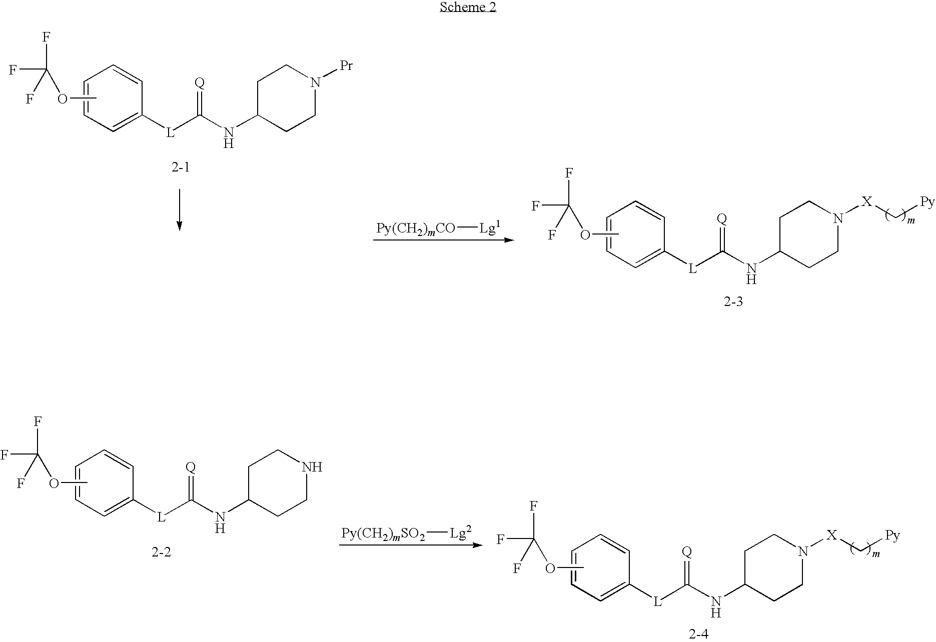

As shown in Scheme 2, Compound 2-1 can be deprotected to the free amino Compound 2-2 under conditions known for deprotecting the particular protecting group used. For example, when Pr is Boc, it can be removed under acidic conditions using an acid, such as HCl or trifluoroacetic acid; when Pr is Cbz, it can be removed under hydrogenation conditions, such as using hydrogen gas in the presence of a catalyst, such as palladium on carbon; when Pr is Fmoc, it can be removed under basic conditions using a base such as piperidine. Compound 2-2 can then react with Py-(CH₂)ₘ—CO-Lg¹ (Lg¹ is OH or a leaving group such as halo) to form the amide Compound 2-3 or react with Py-(CH₂)ₘSO₂-Lg² (Lg² is a leaving group such as halo) to form the sulfonamide Compound 2-4. These reaction conditions are generally known to those of skill in the art.

The urea compounds of this invention can also be prepared according to Scheme 3.

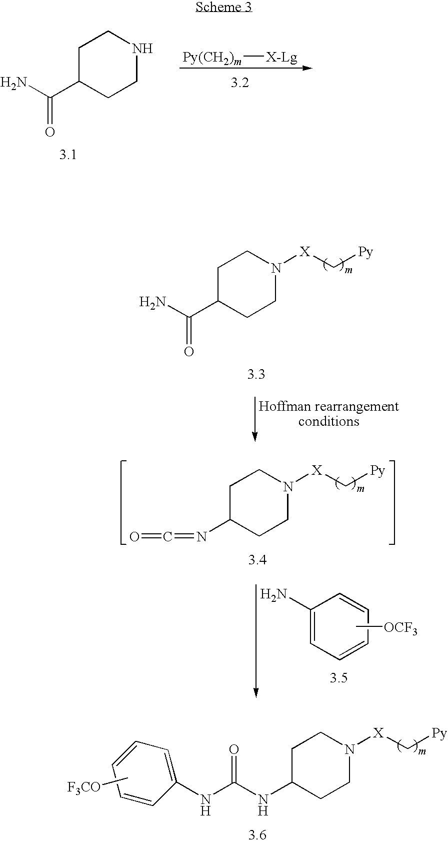

where X, m, and Py are defined herein and Lg is a suitable leaving group.

In Scheme 3, the amino group of Compound 3.1 reacts with Py(CH₂)ₘ—X-Lg, such as an acid chloride or sulfonyl chloride, using conventional conditions. Suitable bases may be used to neutralize possible acid generated. Such bases are well known in the art and include, by way of example only, triethylamine, diisopropylethylamine, pyridine, and the like.

The reaction is typically conducted at a temperature of from about 0 to about 40° C. for a period of time sufficient to effect substantial completion of the reaction which typically occurs within about 1 to about 24 hours. Upon reaction completion, Compound 3.3, can be isolated by conventional conditions such as precipitation, evaporation, chromatography, crystallization, and the like or, alternatively, used in the next step without isolation and/or purification. In certain cases, Compound 3.3 precipitates from the reaction.

Compound 3.3 is then subjected to Hoffman rearrangement conditions to form isocyanate Compound 3.4 under conventional conditions. In certain cases, Hoffman rearrangement conditions comprise reacting with an oxidative agent preferably selected from (diacetoxyiodo)benzene, base/bromine, base/chlorine, base/hypobromide, or base/hypochloride. Specifically, approximately stoichiometric equivalents of Compound 3.3, and, e.g., (diacetoxyiodo)benzene are combined in the presence of a suitable inert diluent such as acetonitrile, chloroform, and the like. The reaction is typically conducted at a temperature of from about 40° C., to about 100° C., and preferably at a temperature of from about 70° C., to about 85° C., for a period of time sufficient to effect substantial completion of the reaction which typically occurs within about 0.1 to about 12 hours. Upon reaction completion, the intermediate isocyanate, Compound 3.4, can be isolated by conventional conditions such as precipitation, evaporation, chromatography, crystallization, and the like.

Alternatively and preferably, this reaction is conducted in the presence of trifluoromethoxyphenyl amine, Compound 3.5, such that upon formation of the isocyanate, Compound 3.4, the isocyanate functionality of this compound can react in situ with the amino functionality of Compound 3.5 to provide for Compound 3.6. In this embodiment, the calculated amount of the intermediate isocyanate is preferably employed in excess relative to the amine and typically in an amount of from about 1.1 to about 1.2 equivalents based on the number of equivalents of the amine employed. The reaction conditions are the same as set forth above and the resulting product can be isolated by conventional conditions such as precipitation, evaporation, chromatography, crystallization, and the like.

In some embodiments, Compound 3.4 is a stable intermediate. In certain cases, Compound 3.4 is formed substantially free from impurities. Hence, Scheme 4 can be run as telescoping reaction processes.

The following compounds in Table 2 were prepared according to one or more the above general schemes and procedures or modifications known in the art. They are provided to illustrate certain aspects of the present invention and to aid those of skill in the art in practicing the invention. These examples are in no way to be considered to limit the scope of the invention.

TABLE 2

| | | Mass | HPLC | M.P. Range |
|---|---|---|---|---|
| Compound # | Structure | [M + 1] | Purity (%) | (° C.) |
| 1 | 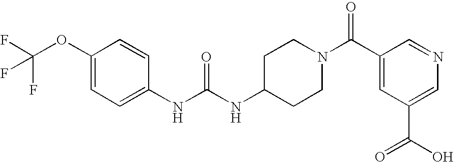 | 453 | 99 | 211-213 |
| 2 | 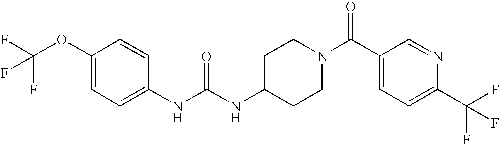 | 477 | 99 | 206-208 |
| 3 | 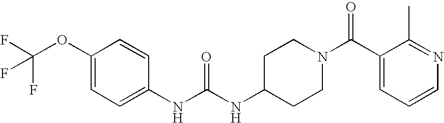 | 423 | 99 | 107-109 |
| 4 | 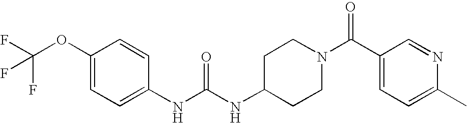 | 423 | 99 | 206-208 |
| 5 | 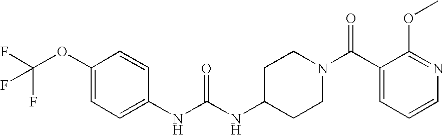 | 439 | 99 | 92-94 |
| 6 | 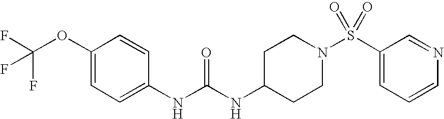 | 445 | 92.8 | 217-219 |
| 7 | 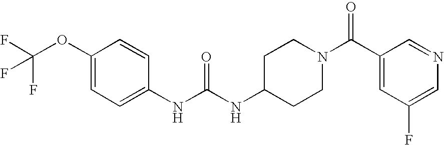 | 427 | 97.5 | 156-157 |
| 8 | 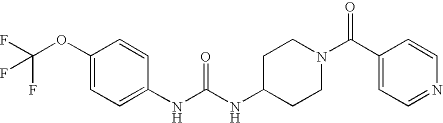 | 409 | 96.8 | 145-147 |
| 9 | 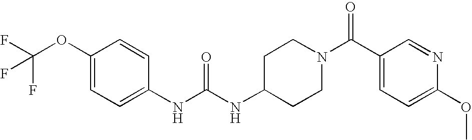 | 439 | 97.9 | 200-202 |
| 10 | 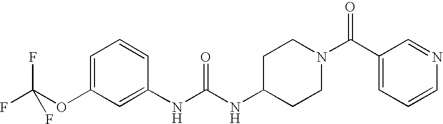 | 409 | 100 | — |
| 11 | 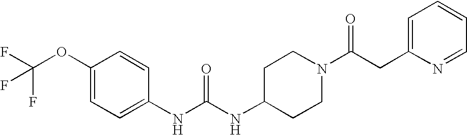 | 423 | 94 | 182-184 |
| 12 | 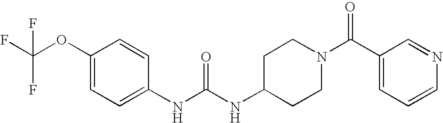 | 409 | 96.9 | 217-222 |
| 13 | 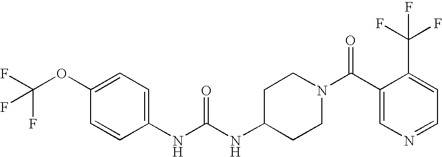 | 477 | 99 | 89-91 |
| 14 | 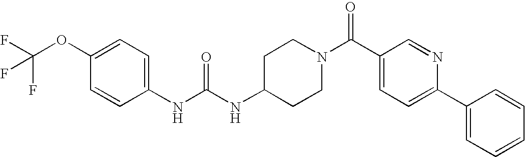 | 485 | 97.1 | 230-232 |
| 15 | 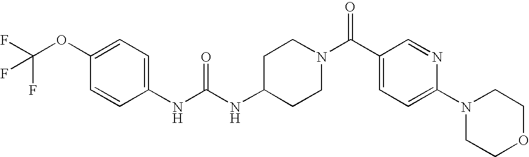 | 494 | 93.5 | 265-268 |
| 16 | 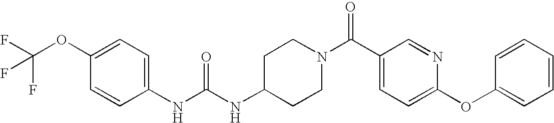 | 501 | 96.8 | 182-185 |
| 17 | 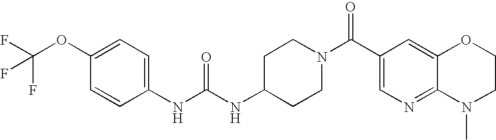 | 480 | 95.4 | 212-214 |
| 18 | 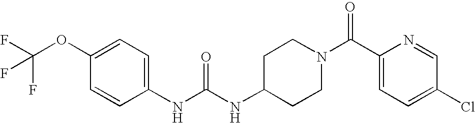 | 443 | 95.2 | 213-215 |

### BIOLOGICAL EXAMPLES

### Example 1

### Fluorescent Assay for Mouse and Human Soluble Epoxide Hydrolase

Recombinant mouse sEH (MsEH) and human sEH (HsEH) were produced in a baculovirus expression system as previously reported. Grant et al., J. Biol. Chem., 268:17628-17633 (1993); Beetham et al., Arch. Biochem. Biophys., 305:197-201 (1993). The expressed proteins were purified from cell lysate by affinity chromatography. Wixtrom et al., Anal. Biochem., 169:71-80 (1988). Protein concentration was quantified using the Pierce BCA assay using bovine serum albumin as the calibrating standard. The preparations were at least 97% pure as judged by SDS-PAGE and scanning densitometry. They contained no detectable esterase or glutathione transferase activity which can interfere with the assay. The assay was also evaluated with similar results in crude cell lysates or homogenate of tissues.

The IC₅₀s for each inhibitor were according to the following procedure:

### Substrate:

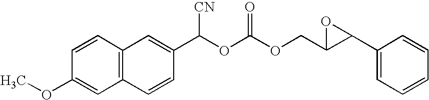

Cyano(2-methoxynaphthalen-6-yl)methyl (3-phenyloxiran-2-yl)methyl carbonate (CMNPC; Jones P. D. et. al.; Analytical Biochemistry 2005; 343: pp. 66-75)

### Solutions:

Bis/Tris HCl 25 mM pH 7.0 containing 0.1 mg/mL of BSA (buffer A)

### CMNPC at 0.25 mM in DMSO.

Mother solution of enzyme in buffer A (Mouse sEH at 6 μg/mL and Human sEH at 5 μg/mL).
Inhibitor dissolved in DMSO at the appropriate concentration.

### Protocol:

In a black 96 well plate, fill all the wells with 150 μL of buffer A.
Add 2 μL of DMSO in well A2 and A3, and then add 2 μL of inhibitor solution in A1 and A4 through A12.
Add 150 μL of buffer A in row A, then mix several time and transfer 150 μL to row B. Repeat this operation up to row H. The 150 μL removed from row H is discarded.
Add 20 μL of buffer A in column 1 and 2, then add 20 μL of enzyme solution to column 3 to 12.
Incubate the plate for 5 minutes in the plate reader at 30° C.
During incubation prepare the working solution of substrate by mixing 3.68 mL of buffer A (4×0.920 mL) with 266 μL (2×133 μL) of substrate solution).
At t=0, add 30 μL of working substrate solution with multi-channel pipette labeled “Briggs 303” and start the reading ([S]_{final}: 5 μM).
Read with ex: 330 nm (20 nm) and em: 465 nm (20 nm) every 30 second for 10 minutes. The velocities are used to analyze and calculate the IC₅₀s.

Table 3 shows the percent inhibition (% Inhibition) of Compounds 1-18 when tested with the assay at 200 or 2000 nM.

TABLE 3

| Compound # | Concentration (nM) | % Inhibition |
|---|---|---|
| 1 | 200 | 94 |
| 2 | 200 | 97 |
| 3 | 200 | 95 |
| 4 | 200 | 97 |
| 5 | 200 | 99 |
| 6 | 200 | 96 |
| 7 | 2000 | 98 |
| 8 | 2000 | 99 |
| 9 | 2000 | 99 |
| 10 | 2000 | 99 |
| 11 | 2000 | 100 |
| 12 | 2000 | 100 |
| 13 | 2000 | 99 |
| 14 | 2000 | 100 |
| 15 | 2000 | 100 |
| 16 | 2000 | 100 |
| 17 | 2000 | 100 |
| 18 | 2000 | 97 |

### FORMULATION EXAMPLES

The following are representative pharmaceutical formulations containing a compound of the present invention.

### Example 1

### Tablet Formulation

The following ingredients are mixed intimately and pressed into single scored tablets.

| Ingredient | Quantity per tablet, mg |
|---|---|
| Compound of the invention | 400 |
| Cornstarch | 50 |
| Croscarmellose sodium | 25 |
| Lactose | 120 |
| Magnesium stearate | 5 |

### Example 2

### Capsule Formulation

The following ingredients are mixed intimately and loaded into a hard-shell gelatin capsule.

| Ingredient | Quantity per tablet, mg |
|---|---|
| Compound of the invention | 200 |
| Lactose, spray-dried | 148 |
| Magnesium stearate | 2 |

### Example 3

### Suspension Formulation

The following ingredients are mixed to form a suspension for oral administration (q.s.=sufficient amount).

| Ingredient | Amount | |
|---|---|---|
| Compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| flavoring | 0.035 | mL |
| colorings | 0.5 | mg |

| | |
|---|---|
| distilled water | q.s to 100 mL |

### Example 4

### Injectable Formulation

The following ingredients are mixed to form an injectable formulation.

| Ingredient | Quantity per tablet, mg |
|---|---|
| Compound of the invention | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per tablet, mg |
|---|---|
| Compound of the invention | 500 mg |
| Witepsol ® H-15 | balance |

What is claimed is:

1. A compound of Formula (I) or pharmaceutically acceptable salt thereof: 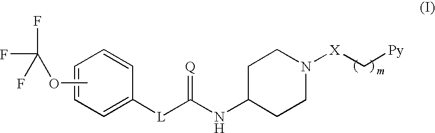
wherein
Q is O or S;
L is —NH—, a covalent bond, or —CR¹R²—; where R¹ and R² are independently hydrogen or alkyl or R¹ and R² together with the carbon atom bound thereto form a C₃-C₆ cycloalkyl ring;
Py is pyridyl or substituted pyridyl;
X is —C(O)—, or —SO₂—; and
m is 0, 1, or 2; and
wherein when m is 0 and Q is O, then X is on the 3- or 4-position of the pyridyl ring.

2. The compound in accordance with claim 1, wherein L is —NH—.

3. The compound in accordance with claim 1, wherein L is —CR¹R²— where R¹ and R² are independently H or alkyl or R¹ and R² together with the carbon atom bound thereto form a C₃-C₆ cycloalkyl ring.

4. The compound in accordance with claim 3, wherein L is —CH₂—.

5. The compound in accordance with claim 1, wherein L is a covalent bond.

6. The compound in accordance with claim 1, wherein X is —C(O)—.

7. The compound in accordance with claim 1, wherein X is —SO₂—.

8. The compound in accordance with claim 1, wherein Q is O.

9. The compound in accordance with claim 1, wherein Q is S.

10. The compound in accordance with claim 1, wherein m is 0.

11. The compound in accordance with claim 1, wherein m is 1.

12. The compound in accordance with claim 1 of Formula (IIa) or (IIb), or pharmaceutically acceptable salt thereof 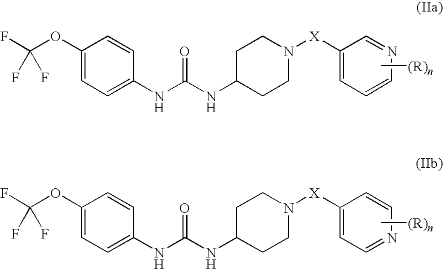
wherein
X is —C(O)—, or —SO₂—;
each R independently is selected from the group consisting of halo, alkyl, substituted alkyl, cycloalkyl, substituted cycloalkyl, aryl, substituted aryl, heterocyclic, substituted heterocyclic, aryloxy, substituted sulfonyl, acylamino, aminocarbonyl, (carboxyl ester)amino, carboxy, carboxyl ester, alkoxy, substituted alkoxy, cyano, and nitro; or two R groups on two adjacent pyridyl carbon atoms join together to form an optionally substituted heterocyclic group fused with the pyridyl ring; and
n is 0, 1, 2, 3, or 4.

13. The compound in accordance with claim 1 of Formula (IIIa) or (IIIb), or pharmaceutically acceptable salt thereof 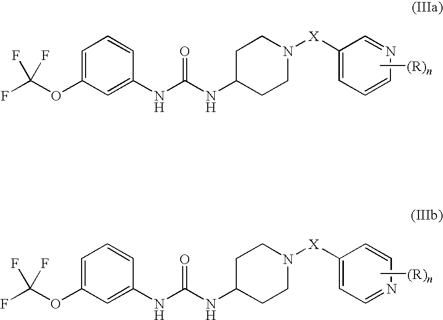
wherein
X is —C(O)—, or —SO₂—;
each R is independently selected from the group consisting of halo, alkyl, substituted alkyl, cycloalkyl, substituted cycloalkyl, aryl, substituted aryl, heterocyclic, substituted heterocyclic, alkoxy, substituted alkoxy, aryloxy, substituted sulfonyl, acyl, acylamino, aminocarbonyl, (carboxyl ester)amino, carboxyl, carboxyl ester, cyano, and nitro; or two R groups on two adjacent pyridyl carbon atoms join together to form an optionally substituted heterocyclic group fused with the pyridyl ring; and
n is 0, 1, 2, 3, or 4.

14. The compound in accordance with claim 1 of Formula (IVa), (IVb) or (IVc), or pharmaceutically acceptable salt thereof 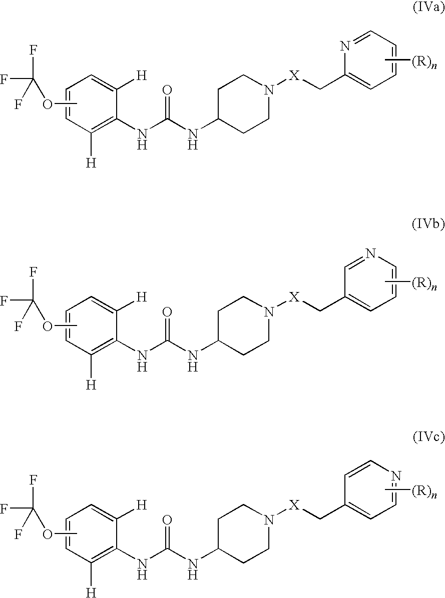
wherein
X is —C(O)—, or —SO₂—;
each R is independently selected from the group consisting of halo, alkyl, substituted alkyl, cycloalkyl, substituted cycloalkyl, aryl, substituted aryl, heterocyclic, substituted heterocyclic, alkoxy, substituted alkoxy, aryloxy, substituted sulfonyl, acyl, acylamino, aminocarbonyl, (carboxyl ester)amino, carboxy, carboxyl ester, cyano, and nitro; or two R groups on two adjacent pyridyl carbon atoms join together to form a heterocyclic group fused with the pyridyl ring; and
n is 0, 1, 2, 3, or 4.

15. The compound in accordance with claim 12, 13, or 14, wherein X is —SO₂—.

16. The compound in accordance with claim 12, 13, or 14, wherein X is —C(O)—.

17. The compound in accordance with claim 12, 13, or 14, wherein n is 0.

18. The compound in accordance with claim 12, 13, or 14, wherein n is 1 or 2, and each R is independently selected from the group consisting of halo, alkyl, substituted alkyl, alkoxy, substituted alkoxy, carboxy, aryloxy, aryl, heterocyclic, and nitro.

19. The compound in accordance with claim 12, 13, or 14, wherein R is selected from the group consisting of fluoro, chloro, methyl, trifluoromethyl, methoxy, trifluoromethoxy, phenoxy, phenyl, morpholino, and carboxy.

20. The compound in accordance with claim 12, 13, or 14, wherein two R groups on two adjacent carbon atoms join together to form an optionally substituted heterocyclic ring fused with the pyridyl ring.

21. The compound in accordance with claim 1 selected from the group consisting of 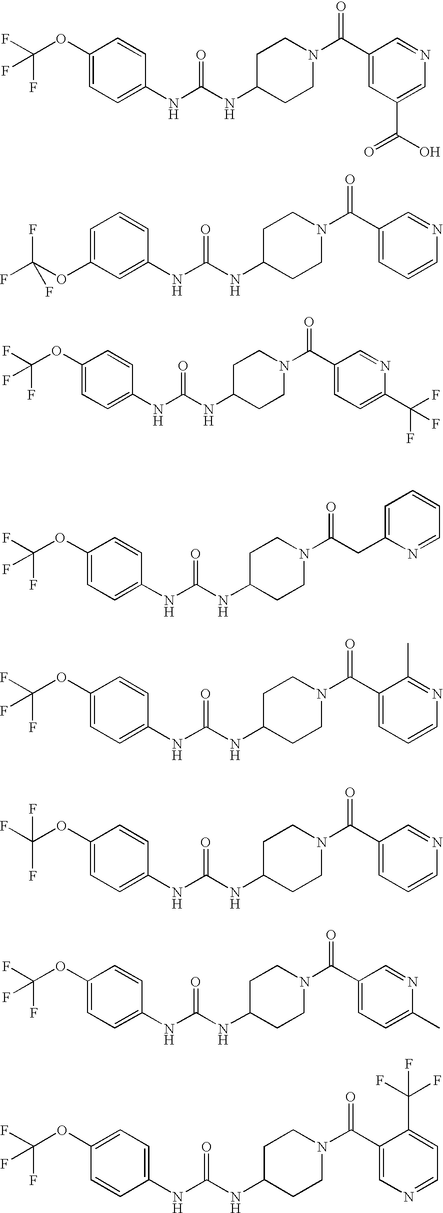 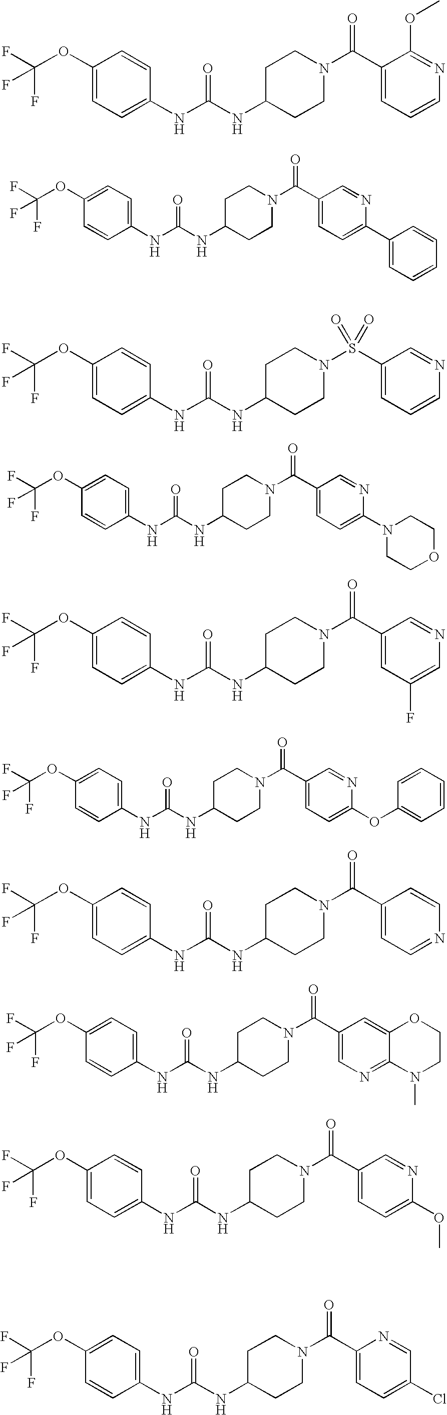
or a pharmaceutically acceptable salt thereof.

22. A pharmaceutical composition comprising a pharmaceutically acceptable carrier and a therapeutically effective amount of a compound of claim 1 or a pharmaceutically acceptable salt thereof for treating a soluble epoxide hydrolase mediated disease.

23. A method for treating a soluble epoxide hydrolase mediated disease, said method comprising administering to a patient a pharmaceutical composition comprising a pharmaceutically acceptable carrier and a therapeutically effective amount of a compound of claim 1 or a pharmaceutically acceptable salt thereof.

24. The method of claim 23 wherein the disease is selected from the group consisting of hypertension, inflammation, adult respiratory distress syndrome, diabetic complications, end stage renal disease, metabolic syndrome, Raynaud syndrome, arthritis, obstructive pulmonary disease, interstitial lung disease, and asthma.

25. A method for inhibiting a soluble epoxide hydrolase, comprising contacting the soluble epoxide hydrolase with an effective amount of a compound of claim 1 or a pharmaceutically acceptable salt thereof.
